(19) United States
(12) Patent Application Publication
(10) Pub. No.: US 2008/0095816 A1
(43) Pub. Date: Apr. 24, 2008

(54) Compositions and Devices Comprising Silicone and Specific Polyphosphazenes

(75) Inventors: Thomas A. Gordy, Newnan, GA (US); Neng S. Ung, Lincolnshire, IL (US); Ulf Fritz, Hirschhorn (DE); Olaf Fritz, Hirschhorn (DE); Ronald Wojcik, Canton, GA (US)

Correspondence Address: SUTHERLAND ASBILL & BRENNAN LLP, 999 PEACHTREE STREET, N.E., ATLANTA, GA, 30309, US

(73) Assignee: CELONOVA BIOSCIENCES, INC., Newnan, GA (US)

(21) Appl. No.: 11/869,960

(22) Filed: Oct. 10, 2007

Related U.S. Application Data
(60) Provisional application No. 60/828,833, filed on Oct. 10, 2006.

(51) Int. Cl. A61F 2/00

(52) U.S. Cl. 424/422

(57) ABSTRACT

The present invention relates to compositions and medical devices comprising both polyorganosiloxane and polyphosphazene compounds. When incorporated into or onto medical devices, these compositions reduce cell encrustation on the device and reduce the severity of thrombosis when the devices are in contact with body fluids, and impart anti-rejection properties to the device.

### CROSS-REFERENCE TO RELATED APPLICATIONS

This application claims the benefit of U.S. Provisional Patent Application No. 60/828,833, filed Oct. 10, 2006, the entirety of which is hereby incorporated by reference.

### FIELD OF THE INVENTION

The present invention relates to medical devices and compositions that convey beneficial and/or improved properties to the medical devices by, for example, reducing cellular or bacterial adhesion and/or proliferation, reducing organic or inorganic encrustation, reducing the risk of thrombosis, or improving the biological acceptance (anti-rejection properties) of the medical device within the host subject.

### BACKGROUND OF THE INVENTION

Contemporary medical procedures often require medical devices to be implanted into a human or animal subject and remain in periodic or continuous contact with endogenous or exogenous tissue and body fluids over extended time periods. Tubing is a common example of an implantable device and has numerous applications in medical procedures. For example, tubing can include fluid and drug delivery tubing, external feeding tubing, wound or fluid drain tubing, and catheters, all of which are required to survive continual contact with the subject's tissue and fluids. However, the presence of such medical devices in a human or animal body, or any device that otherwise contacts tissue, fluids, or organs, can induce undesirable reactions such as inflammation, infection, thrombosis, cellular and bacterial adhesion, proliferation and/or overexpression of growth, organic, or inorganic encrustation (matter buildup), restenosis, and the like. Such devices also can result in the proliferation of cell growth that can occlude passageways, including those passageways created by the tube itself.

Implantable devices other than tubes are also used in contemporary medical procedures. For example, implants for the chin, cheek, nose, malar, pectoralis, calf, breast, and buttock usually are made of soft or semi-firm/fluid silicone rubber which is inserted into a region of the body to augment, (bio)mechanically stabilize, or reconstruct that region of the body. In breast augmentation surgery, a shell is inserted into a cavity and the shell is either pre-filled with a fluid or filled with fluid after insertion. While the actual materials used to manufacture these devices have changed over the past several years, silicone is still a fundamental material used in or for such devices.

Silicone is a useful and popular material for the synthesis of many medical implants. However, the use of silicone is not without risk and adverse effects have been associated with the use of silicone. In animal models where silicone has been used as a bone graft, silicone has been associated with prolonged local fluid accumulation and resorption of the underlying bone, requiring the patient to undergo additional corrective surgery. Silicone catheters have been associated with encrustation and blockage of the catheter which is related to infection of the urinary tract and urethritis, which can develop within a relatively short time post-catheterization. Additionally, silicone has been associated with a high inflammatory index even in the absence of bacterial infections. When bacteria are present, silicone has a higher likelihood of purulent infection than other materials. Silicones are also now well-recognized inducers of localized granulomatous inflammation. See Cole, P.; Zackson, D. A.; Am. J. Clin. Pathol., 1990, January, 93(1), 148-52. Additionally, silicones are relatively acid-sensitive. For example, stomach acids are known to have a detrimental effect on silicones. Furthermore, after exposure to a biological environment, including prolonged exposure to biological fluids, loss of mechanoelastic flexibility and increased rigidity may be observed. In addition, reduced biocompatibility may result due to plasticizers and lubricating agents, such as oligomeric siloxanes and long chain fatty acids, which can surface-migrate and leach from the implant over time, thereby causing an undesired biological response.

Because silicone materials are commonly used in implantable medical devices, there is a need for some method to mediate or remedy the adverse effects of silicone. This need is widespread, because silicone materials are used in devices that include medical tubing, dressings, expanders, drainage tubes, pump parts, T-drains, intraocular lenses, contact lenses, skin expanders, mammary implants, tracheostoma vents, comforters, membrane dressings, foils, insulation such as insulation for pacemaker electrodes, joint replacements, vascular implants, pins, clips, valves including heart valves, shunts, screws, plates, grafts, stents, implants, pacemaker parts, defibrillator parts, electrode parts, surgical devices, surgical instruments, artificial membranes or structures, parts of artificial organs or tissues, and the like. Therefore, any compounds, compositions, treatments, and/or methods that could help reduce the adverse effects of silicone when used in medical devices are needed.

### SUMMARY OF THE INVENTION

The present invention provides medical devices for introduction into a human or animal body or organ, or which has contact with tissue or fluids of the human or animal body or organ, comprising a polyorganosiloxane (also called a “silicone”) and one or more specific polyphosphazenes. This combination of materials has been found to render the medical device more biocompatible, more lubricious, anti-microbial, and anti-thrombogenic.

The medical device and methods encompassing the device are not limited as to the exact disposition of the polyorganosiloxane and polyphosphazene components, for example, the polyorganosiloxane can be coated (or layered) with, reacted with, blended (or mixed) with, grafted to, bonded to, crosslinked with, copolymerized with, coated and/or reacted with an intermediate layer that is coated and/or reacted with, or combined with the polyphosphazene in any manner. Further, the polyphosphazenes of the present invention can be combined with a polyorganosiloxane and the combination can be coated on a device or a surface such that the polyphosphazene and polyorganosiloxane are coated at substantially the same time. All these aspects are encompassed by the disclosure that any material includes or comprises a polyorganosiloxane and a specific polyphosphazene, or by the disclosure that a particular polyphosphazene is added to a polyorganosiloxane. As used herein, polyorganosiloxanes are also referred to as silicone, polysiloxane, or simply polymerized siloxanes.

In another aspect, this disclosure provides a medical device comprising a polyorganosiloxane in combination with a specific polyphosphazene or derivatives or analogs thereof represented by formula I:

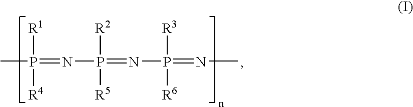

wherein n is 2 to ∞; and R¹ to R⁶ are groups which are each selected independently from alkyl, aminoalkyl, haloalkyl, thioalkyl, thioaryl, alkoxy, haloalkoxy, aryloxy, haloaryloxy, alkylthiolate, arylthiolate, alkylsulphonyl, alkylamino, dialkylamino, heterocycloalkyl comprising one or more heteroatoms selected from nitrogen, oxygen, sulfur, phosphorus, or a combination thereof, or heteroaryl comprising one or more heteroatoms selected from nitrogen, oxygen, sulfur, phosphorus, or a combination thereof. In one aspect, for example, the polyorganosiloxane can constitute part, such as a coating, or all of the medical device, and the polyphosphazene can be included in the device with the polyorganosiloxane in any manner. The present invention also provides a method for making a medical device more biocompatible, more lubricious, anti-microbial, and anti-thrombrogenic, comprising adding to the polyorganosiloxane a polyphosphazene. In addition, the polyphosphazene can be used in combination with or without an adhesion promoter, whether monomeric, oligomeric or polymeric, a tie layer, a surfactant, a dispersing agent, a filling agent, a stabilizer, or any other agent targeted at improving the interfacial compatibility and/or stability between the polyphosphazene and polyorganosiloxane compounds when contacting each other.

In another aspect, this disclosure provides a medical device comprising a polyorganosiloxane and a poly[bis(2,2,2-trifluoroethoxy)phosphazene]. Further, this invention provides compositions comprising silicones and particular polyphosphazenes, wherein the polyphosphazene is poly[bis(trifluoro-ethoxy)phosphazene], also called poly[bis(2,2,2-trifluoroethoxy)phosphazene].

### BRIEF DESCRIPTION OF THE DRAWINGS

FIG. 1 is a scanning electron microscope (SEM) image at 1600× magnification of a Silastic® Foley catheter that was treated with poly[bis(2,2,2-trifluoroethoxy)]-phosphazene, following a 3-day incubation in artificial urine containing E. coli.

FIG. 2 is a scanning electron microscope (SEM) image at 550× magnification of a Silastic® Foley catheter that was not treated with any polyphosphazene, following a 3-day incubation in artificial urine containing E. coli.

FIG. 3 is a scanning electron microscope (SEM) image at 1600× magnification of a Silastic® Foley catheter that was not treated with any polyphosphazene, following a 3-day incubation in artificial urine containing E. coli.

### DETAILED DESCRIPTION OF THE INVENTION

This invention relates to a medical device for introduction into a human or animal body or organ, or which has contact with tissue or fluids of the human or animal body or organ, comprising a polyorganosiloxane in combination with a polyphosphazene, or in alternative language, comprising a polyorganosiloxane to which a polyphosphazene has been added.

In one aspect, this invention provides a device comprising a particular polyphosphazene or derivatives thereof in combination with a polyorganosiloxane. While not intending to be bound by theory, by describing the polyphosphazene “in combination” with the polyorganosiloxane, it is intended to reflect, without limitation, that the polyphosphazene is in contact with the polyorganosiloxane, or the polyphosphazene is in contact with an intermediate component which is in contact with the polyorganosiloxane. Intermediate components include materials such as the adhesion promoters, tie layers, transitional materials, interposing layers, and the like, as disclosed herein. As used herein, the term “in contact” includes any chemical or physical interaction between or among the components or layers. For example, a polyphosphazene in contact with a polyorganosiloxane is intended to include any of the combinations of a silicone and the particular polyphosphazene disclosed herein, including any copolymer thereof (random, alternating, block, graft, comb, star, dendritic, and the like), interpenetrating networks between the silicone and the polyphosphazene, blends, or other chemical or physical interactions. Similarly, by describing the polyphosphazene as being in contact with an intermediate component which is in contact with the polyorganosiloxane, it is intended to include any type of chemical reaction, bonding, ionic and/or electrostatic interaction, or any type of physical and or chemical process, by which all these components achieve their interaction. It is to be understood that any device comprising a polyphosphazene in combination with a polyorganosiloxane can include any of these contact interaction types, including any combination thereof, and/or include contact interactions not readily identified as falling into one type or the other, but rather are situated along a continuum of interaction modes (as measured by parameters such as bond energies, van der Waals interactions, ionic interactions, electrostatic interactions, Lewis acid/base complex formation, and the like) between these two.

Polyorganosiloxanes. In one aspect, the polyorganosiloxane constitutes part of the medical device, such as a coating, although in some embodiments the medical device is prepared from the polyorganosiloxane itself (forming the bulk material). The terms polyorganosiloxane, polysiloxane, or silicone refers to a general category of synthetic polymers whose backbone is made of repeating silicon to oxygen bonds. In addition to their links to oxygen to form the polymeric backbone chain, the silicon atoms are also bonded to side groups, typically organic groups. In one aspect, the organic side groups comprise methyl groups. One common silicone is characterized by having two methyl groups bonded to each silicon atom in the polymeric chain; therefore, this silicone is made of repeating [—O—SiMe₂-] units. This silicone is termed polydimethylsiloxane (or dimethylpolysiloxane), commonly abbreviated as PDMS.

However, many other polyorganosiloxanes may be used in this invention. For example, suitable polyorganosiloxanes include, but are not limited to, those in which any of the following groups may be bonded to the silicon in a polyorganosiloxane structure: alkyl, aryl, alkyloxy (alkoxy), aryloxy, haloalkyl, haloaryl, haloalkoxy, haloaryloxy, alkenyl, alkynyl, alkyl- or aryl-ether groups, alkyl- or aryl-ester groups, O-heterocyclic groups, N-heterocyclic groups, and other heterocyclic variants thereof, and combinations thereof, including any isomer thereof, wherein any group can have up to about 20 carbon atoms. Examples of specific groups that are useful include, but are not limited to, methyl, ethyl, n-propyl, iso-propyl, n-butyl, t-butyl, phenyl, tolyl, xylyl, benzyl, imidazolyl, vinyl, vinylbenzyl, methoxy, ethoxy, n-propoxy, iso-propoxy, chlorophenyl, fluorophenyl, trifluoromethyl, trifluoroethyl, trifluoropropyl, hexafluoro-isopropyl, acetic acid esters, formic acid esters and the like, including any combination thereof. Thus, potentially hydrolyzable groups containing methoxy, ethoxy, propoxy, ether or acetic or formic acid esters, attached indirectly as titanoate or zirconate, or directly to the siloxane backbone and the like, are often substituted for the methyl groups along the chain, providing for the corresponding homo- or copolymeric siloxane formulations or blends with desired properties generally known and used in the art. Substituents such as these may be substituted for some or all the methyl groups in a polydimethylsiloxane structure, providing for the corresponding homopolymeric or copolymeric siloxane formulations or blends with the desired properties, as known by one of ordinary skill. Other groups may be substituted for some or all of the methyl groups in a polydimethylsiloxane structure, such as phenyl, ethyl, vinyl, allyl, and the like, in which such groups can be partially or totally halogenated. Examples of halogenated groups include, but are not limited to, pentafluorophenyl, trifluoroethyl, or trifluoromethylphenyl groups. Moreover, copolymeric siloxane formulations or blends with the desired properties are known and used in the art.

The particular polysiloxane or “silicone” that can be used in this invention is not limiting. Rather, any silicone that is used, or can be used, in a medical device, including any device that is adapted for introduction into a human or animal body, organ, vessel, or cavity, or which has contact with tissue or fluids (liquids and/or gases) of the human or animal body or organ, is encompassed by this invention. Further, this disclosure is applicable to any silicone classified according to the principal industrial classifications of silicone rubbers, for example, High Temperature Vulcanizing (HTV) silicones, Room Temperature Vulcanizing (RTV) silicones, and even Liquid Silicone Rubbers (LSR) can be employed in this invention. Moreover, any silicone rubber according to the ASTM D1418 classifications for silicone rubber can be employed, examples of which are provided in Table 1.

TABLE 1
ASTM D1418 Classifications for Silicone Rubber

| Class | Description |
|---|---|
| MQ | Silicone rubbers having only methyl groups on the polymer |
| | chain (polydimethylsiloxanes) |
| VMQ | Silicone rubbers having methyl and vinyl substitutions on the |
| | polymer chain |
| PMQ | Silicone rubbers having methyl and phenyl substitutions on |
| | the polymer chain |
| PVMQ | Silicone rubbers having methyl, phenyl and vinyl |
| | substitutions on the polymer chain |
| FVMQ | Silicone rubbers having fluoro, methyl and vinyl substitutions |
| | on the polymer chain |

Commonly used terms for these various compounds include silicone, silicone-elastomers (including, but not limited to high-consistency elastomers, liquid-silicone rubbers, low-consistency silicones, and adhesives), silicone-rubber, fluorosilicones, polymers of fluorosilicones, dimethylsilicones, phenyl-containing silicones, vinyl-containing silicones, substituted silicones, silicone resins, blends of silicone resins and elastomers, silicone gels, silicone liquid elastomers, polysiloxanes, and other siloxanes which are solid at room temperature. All such materials are encompassed by this invention. The terminal group on the polymer can also comprise a trimethylsilyloxy terminus or termini, but the methyl groups on these ends can also be substituted for other groups or atoms. The exact type of silicone is not limited in the present invention as the polyphosphazene that is added to the silicone works efficiently and adds beneficial properties to silicones including, but not limited to, room and heat and chemical and irradiation curable silicone, liquid injection molded silicone, silicone liquid elastomers, condensation curable silicones, addition curable silicones and elastomeric, and resinous silicones. Therefore, further examples of silicones include, but are not limited to, room temperature curable (RTV), moisture-curable, platinum curable, peroxy curable, or more generally, metal and radical-curable silicones.

Additionally, filler materials comprising compounds, or compositions can be added to the silicone. For example, carbon black, titanium oxide, barium sulfate, silica fillers such as fumed silica, or various pigments can be added to the silicone to impart additional properties to the silicone, as understood by one of ordinary skill. For example, filler materials can be used for altering hapticity, for providing properties of inflexibility or flexibility, for changing optical quality such as radiopaqueness, or electro-magnetic properties, or for altering conductivity properties.

Devices. In one aspect, this invention encompasses any device that contains silicone, and provides methods of making devices that comprise silicone, the method comprising combining the silicone and polyphosphazene of the present invention. For example, tubing that is not medical grade tubing is also within the scope of present invention. Other examples comprise various seals, gaskets, bellows, rollers, valves, extruded devices, molded devices, sculpted devices, carved devices, shaped devices, and the like. The underlying material that the device is composed of is not limited, as this invention is applicable to any device that contains silicone. The polyphosphazene that is added to the silicone imparts properties to the silicone that are also beneficial to non-medical uses. For example, the polyphosphazenes of the present invention have and impart a high degree of lubricity as well as non-stick properties, which aid in the transfer of material or fluids within the tubing or over a surface of the device, and reduce frictional wear on components, contacting surfaces, and the surrounding environment. Additionally, the polyphosphazene of the present invention that is added to the silicone-containing device imparts an anti-bacterial property to the device which can decrease the maintenance efforts in keeping the device clean. The device also is not limited to tubing and can be any three-dimensional structure or any two-dimensional surface that comprises silicone. For example, solid structures, sheets, and structures with internal voids that are or are not in communication with the outer environment or with other voids within the structure, or combinations thereof are included in the scope of this disclosure.

In one aspect, it is not necessary that the device or medical device contain only a silicone and a polyphosphazene of the present invention. In certain embodiments of the invention, the device or medical device can comprise a composition of silicone and at least one other compound or material in addition to the polyphosphazene. For example, certain medical devices can comprise compositions comprising silicone and urethane or polyurethane copolymers. Additional compositions comprising silicone include those that also contain polyvinylchloride (PVC), acrylics, vinyls, nylons, polyolefins including polyethylenes and polypropylenes, polyethers, polycarbonates, polyesters, polyamides, polyimides, hydrogels, ionomers, silicone rubbers, thermoplastic rubbers, fluoropolymers, other polysiloxanes, and the like. One skilled in the art will recognize the components of the composition comprising silicone and a polyphosphazene can further include any of those materials listed above or others, including any combination thereof, and it can be applied to surfaces of other materials or be mixed, blended, coated onto, grafted to or bonded to other materials as long as the composition contains a silicone and a polyphosphazene.

In another aspect, the device or medical device also can be one in which the silicone and polyphosphazene encapsulate, are applied to one or more surface of, are internal to, or is otherwise a part of the device or medical device. For example, an internal structure such as a metal plate can be coated with a silicone and that layer of silicone or material comprising silicone subsequently can be coated, grafted, blended, or bonded with or to a polyphosphazene. Alternatively, the internal structure can be coated, blended, grafted, or bonded with a composition comprising silicone and a polyphosphazene of the present invention.

The medical device can be introduced into a human or animal body or organ by any number of techniques. For example, the device can be introduced through invasive procedures such as surgery where an opening is made to the human or animal body, organ, vessel, or cavity, and the device is placed within. Alternatively, the human or animal can ingest the device or the device can be placed within an orifice on the human or animal body, or the device can be at least partially attached to the human or animal body. In addition, the device can otherwise be in contact with tissue or fluids (including liquids and gases) of the human or animal body or organ of the human or animal. For example, the device can comprise a tube in which fluids pass and the tube can deliver the fluid to the human or animal without the tube being inserted into the human or animal, such as any extracorporeal device delivering and/or transporting fluids into or out of the subject's body. An additional example comprises a medical device such as a valve that controls the passage or flow of a gas or a fluid where the valve can be inserted into the human or animal body or be placed external to the human or animal body. The exact placement of the device is not limited, as one aspect of this invention is the combination of the silicone-based or silicone-containing device with a polyphosphazene of the present invention, whereby the polyphosphazene imparts beneficial features to the silicone or silicone-containing device.

Polyphosphazenes The device or medical device comprising silicone and a polyphosphazene typically comprises a particular polyphosphazene or derivatives thereof having the following general formula I:

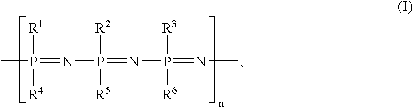

wherein n is 2 to ∞; and R¹ to R⁶ are groups which are each selected independently from alkyl, aminoalkyl, haloalkyl, thioalkyl, thioaryl, alkoxy, haloalkoxy, aryloxy, haloaryloxy, alkylthiolate, arylthiolate, alkylsulphonyl, alkylamino, dialkylamino, heterocycloalkyl comprising one or more heteroatoms selected from nitrogen, oxygen, sulfur, phosphorus, or a combination thereof or heteroaryl comprising one or more heteroatoms selected from nitrogen, oxygen, sulfur, phosphorus, or a combination thereof. Thus, the residues R¹ to R⁶ are each independently variable and therefore can be the same or different. By indicating that n can be as large as ∞ in formula I, it is intended to specify values of n that encompass polyphosphazene polymers that can have an average molecular weight of up to about 75 million Daltons. For example, in one aspect, n can vary from at least about 40 to about 100,000. In another aspect, by indicating that n can be as large as ∞ in formula I, it is intended to specify values of n from about 4,000 to about 50,000, more preferably, n is about 7,000 to about 40,000 and most preferably n is about 13,000 to about 30,000.

In another aspect of this invention, the polymer used to prepare the devices disclosed herein has a molecular weight based on the above formula, which can be a molecular weight of at least about 70,000 g/mol, more preferably at least about 1,000,000 g/mol, and still more preferably a molecular weight of at least about 3×10⁶ g/mol to about 20×10⁶ g/mol. Most preferred are polymers having molecular weights of at least about 10,000,000 g/mol.

In one aspect of this invention, the polyphosphazene is poly[bis(2,2,2-trifluoroethoxy)phosphazene] or a fluorinated alkoxide analog thereof. The preferred poly[bis(trifluoroethoxy)phosphazene] polymer is made up of repeating monomers represented by the formula IA shown below: 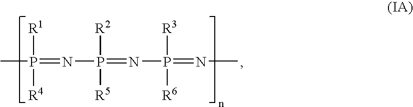
wherein R¹ to R⁶ are all trifluoroethoxy (OCH₂CF₃) groups, and wherein n may vary from at least about 100 to larger molecular weight lengths. For example, n is from about 4,000 to about 500,000, or from about 4,000 to about 3,000. In one aspect, n is from about 13,000 to about 30,000. Alternatively, one may use analogs of this polymer in the preparation of the devices of the invention. The term “analogs” is meant to refer to polymers made up of monomers having the structure of formula IA but where one or more of the R¹ to R⁶ functional group(s) is replaced by a different functional group(s), but where the biological inertness of the polymer is not substantially altered. Exemplary functional groups include ethoxy (OCH₂CH₃), 2,2,3,3,3-pentafluoropropyloxy (OCH₂CF₂CF₃), 2,2,2,2′,2′,2′-hexafluoroisopropyloxy (OCH(CF₃)₂), 2,2,3,3,4,4,4-heptafluorobutyloxy (OCH₂CF₂CF₂CF₃), 3,3,4,4,5,5,6,6,7,7,8,8,8-tridecafluorooctyloxy (OCH₂(CF₂)₇CF₃), 2,2,3,3-tetrafluoropropyloxy (OCH₂CF₂CHF₂), 2,2,3,3,4,4-hexafluorobutyloxy (OCH₂CF₂CF₂CF₃), 3,3,4,4,5,5,6,6,7,7,8,8-dodecafluorooctyloxy (OCH₂(CF₂)₇CHF₂), and the like. Further, in some embodiments, 1% or less of the R¹ to R⁶ groups may be alkenoxy groups, a feature that may assist in crosslinking to provide a more elastomeric phosphazene polymer. In this aspect, alkenoxy groups include, but are not limited to, OCH₂CH═CH₂, OCH₂CH₂CH═CH₂, allylphenoxy groups, and the like, including combinations thereof.

In another aspect, by indicating that n can be as large as ∞ in formulas I or IA, it is intended to specify values of n that encompass polyphosphazene polymers in which the molecular weight is at least about 70,000 g/mol. In another aspect, n can be selected such that the average molecular weight is at least about 1,000,000 g/mol. Further, n can be selected such that the average molecular weight is at least about 10,000,000 g/mol. In yet another aspect, a useful range of average molecular weights is from about 7×10⁶ g/mol to about 25×10⁶ g/mol.

The pendant side groups or moieties (also termed “residues”) R¹ to R⁶ are each independently variable and therefore can be the same or different. Further, R¹ to R⁶ can be substituted or unsubstituted. The alkyl groups or moieties within the alkoxy, alkylsulphonyl, dialkylamino, and other alkyl-containing groups can be, for example, straight or branched chain alkyl groups having from 1 to 20 carbon atoms, it being possible for the alkyl groups to be further substituted, for example, by at least one halogen atom, such as a fluorine atom or other functional group such as those noted for the R¹ to R⁶ groups above. By specifying alkyl groups such as propyl or butyl, it is intended to encompass any isomer of the particular alkyl group.

In one aspect, examples of alkoxy groups include, but are not limited to, methoxy, ethoxy, propoxy, and butoxy groups, and the like, which can also be further substituted. For example the alkoxy group can be substituted by at least one fluorine atom, with 2,2,2-trifluoroethoxy constituting a useful alkoxy group. In another aspect, one or more of the alkoxy groups contains at least one fluorine atom. Further, the alkoxy group can contain at least two fluorine atoms or the alkoxy group can contain three fluorine atoms. For example, the polyphosphazene that is combined with the silicone can be poly[bis(2,2,2-trifluoroethoxy)phosphazene]. Alkoxy groups of the polymer can also be combinations of the aforementioned embodiments wherein one or more fluorine atoms are present on the polyphosphazene in combination with other groups or atoms.

In one aspect, for example, at least one of the substituents R¹ to R⁶ can be an unsubstituted alkoxy substituent, such as methoxy (OCH₃), ethoxy (OCH₂CH₃) or n-propoxy (OCH₂CH₂CH₃). In another aspect, for example, at least one of the substituents R¹ to R⁶ is an alkoxy group substituted with at least one fluorine atom. Examples of useful fluorine-substituted alkoxy groups R¹ to R⁶ include, but are not limited to OCF₃, OCH₂CF₃, OCH₂CH₂CF₃, OCH₂CF₂CF₃, OCH(CF₃)₂, OCCH₃(CF₃)₂, OCH₂CF₂CF₂CF₃, OCH₂(CF₂)₃CF₃, OCH₂(CF₂)₄CF₃, OCH₂(CF₂)₅CF₃, OCH₂(CF₂)₆CF₃, OCH₂(CF₂)₇CF₃, OCH₂CF₂CHF₂, OCH₂CF₂CF₂CHF₂, OCH₂(CF₂)₃CHF₂, OCH₂(CF₂)₄CHF₂, OCH₂(CF₂)₅CHF₂, OCH₂(CF₂)₆CHF₂, OCH₂(CF₂)₇CHF₂, and the like.

Examples of alkylsulphonyl substituents include, but are not limited to, methylsulphonyl, ethylsulphonyl, propylsulphonyl, and butylsulphonyl groups. Examples of dialkylamino substituents include, but are not limited to, dimethyl-, diethyl-, dipropyl-, and dibutylamino groups. Again, by specifying alkyl groups such as propyl or butyl, it is intended to encompass any isomer of the particular alkyl group.

Exemplary aryloxy groups include, for example, compounds having one or more aromatic ring systems having at least one oxygen atom, non-oxygenated atom, and/or rings having alkoxy substituents, it being possible for the aryl group to be substituted for example by at least one alkyl or alkoxy substituent defined above. Examples of aryloxy groups include, but are not limited to, phenoxy and naphthoxy groups, and derivatives thereof including, for example, substituted phenoxy and naphthoxy groups.

The heterocycloalkyl group can be, for example, a ring system which contains from 3 to 10 atoms, at least one ring atom being a nitrogen, oxygen, sulfur, phosphorus, or any combination of these heteroatoms. The heterocycloalkyl group can be substituted, for example, by at least one alkyl or alkoxy substituent as defined above. Examples of heterocycloalkyl groups include, but are not limited to, piperidinyl, piperazinyl, pyrrolidinyl, and morpholinyl groups, and substituted analogs thereof.

The heteroaryl group can be, for example, a compound having one or more aromatic ring systems, at least one ring atom being a nitrogen, an oxygen, a sulfur, a phosphorus, or any combination of these heteroatoms. The heteroaryl group can be substituted for example by at least one alkyl or alkoxy substituent defined above. Examples of heteroaryl groups include, but are not limited to, imidazolyl, thiophene, furane, oxazolyl, pyrrolyl, pyridinyl, pyridinolyl, isoquinolinyl, and quinolinyl groups, and derivatives thereof.

Preparation of Devices Comprising Silicone and Polyphosphazene. The medical device and methods encompassing the device are not limited as to the exact disposition of the polyorganosiloxane and polyphosphazene components, nor by the manner in which the polyorganosiloxane and polyphosphazene are combined, nor by any type of interaction or bonding mechanism that might occur between these components. In general terms, this disclosure provides for a device comprising a polyorganosiloxane in combination with a polyphosphazene, as provided herein.

The following methods of preparing devices and combining the polyorganosiloxane and polyphosphazene components are therefore not limiting, but provided as exemplary. For example, the polyorganosiloxane can be coated with, blended with, mixed with, grafted to, bonded to, layered on, or combined with in any manner. As used herein, all these aspects are encompassed by the disclosure that a polyphosphazene is added to or combined with a polyorganosiloxane, or by the disclosure that any material includes or comprises a polyorganosiloxane and a polyphosphazene. For example, in one aspect, the polyphosphazene can be added to the silicone comprising the device or medical device by adding the polyphosphazene to one or more surfaces of the silicone. For example, the polyphosphazene can be added to (coated, blended, grafted, bonded onto, and the like) an outer surface of the silicone, an inner surface of the silicone, within the body of the silicone or parts thereof, or any combination thereof. Further, the polyphosphazene can be added to more than one surface of the silicone. For example, a silicone tube can be coated, blended, grafted, bonded, and the like, on the outer surface of the tube, the inner surface of the tube, or both the inner and outer surface of the tube. For inner surfaces of a device comprising silicone that are not in fluid communication with an outer surface of the device or those inner surfaces that are encapsulated within the device, the inner surface can be coated, blended, grafted, bonded, and the like, during manufacture during a period where the inner surface is not encapsulated. Alternatively, the inner surface can be coated, blended, grafted, bonded, and the like, with the polyphosphazene by introducing an opening into the device where the polyphosphazene can be coated, blended, grafted, bonded, and the like, onto the inner surface followed by sealing of the opening such that the coated, blended, grafted, or bonded inner surface is now encapsulated. Alternatively, a device that has been coated, blended, grafted, or bonded with a silicone can also or subsequently be additionally coated on the silicone with a polyphosphazene. For example, a valve that comprises a silicone can have one or more surfaces of the valve coated, blended, grafted, or bonded with a polyphosphazene. The polyphosphazene added to a surface of the valve can aid in the flow of gases or fluids past the valve due to the lubricious nature of the polyphosphazene surface.

In a further aspect, when a polyphosphazene of the present invention is added to (coated, blended, grafted, bonded onto, and the like) a surface of the silicone, this combination also may provide for a barrier interface, preventing or regulating the migration of compounds, liquids, or gases into or out of the siloxane body or onto its surface, thereby preventing or regulating in a controlled fashion, respectively, the leakage or loss of these agents. Examples of agents whose migration can be controlled include fillers, stabilizers, pigments, colors, dyes, lakes, surfactants, antistatic agents, lubricating agents, separating agents, pharmaceutical agents, and the like, including combinations thereof. Hence, in one aspect, the combination of a silicone body with a polyphosphazene coating may aid in reducing biodegradation by controlling compound leaching from the silicone body placed within a biological environment. This feature may increase device longevity and/or biostability and help reduce the unfavorable effects of the body-surface interaction. In another aspect, this feature also may prevent the re-fusing or re-welding of silicone surfaces when in close proximity or contact with each other, an effect that is known to the art. The polyphosphazene further provides a surface that resists bacterial growth, exhibits reduced plasma protein adsorption, reduced platelet adhesion, and enhances biocompatibility of the device.

Depending on the processing methods and specific polymer materials, the aforementioned techniques can generate any number of polysiloxane-polyphosphazene structures. In this aspect, for example, the disclosed methods can provide the combination of polysiloxane-polyphosphazene in the form of a combination of homopolymers, copolymers, grafted copolymers, crosslinked structures, and/or interpenetrating networks, and the like. For example, the methods disclosed herein can generate homogeneously structured, indistinguishable intrinsic composite polymer networks, or heterogeneously structured copolymers, in which the different polymer phases form distinguishable, separated domains with a nano-, meso-, or microstructure. In another aspect, for example, the disclosed techniques can generate extrinsic macroscopically distinguishable two- or three-dimensionally linked interfacial polymer phases, such as multilayered structures that impart their specific properties to the composite device as intended by the specific application for the device. It is understood that each type of polymer network can affect the mechanical and surface properties of the polymer mixture and impart a range of desired properties for the desired application for the device.

The polyphosphazene coating can be applied by any number of techniques. In one aspect, for example, the polyphosphazene of the present invention can be applied to the silicone by dipping the silicone in a solution of the polyphosphazene. Thus, solvent evaporation rates, concentration, type of solvent, the specific polyphosphazene, polyphosphazene concentration regime, the specific silicone used, the solvent susceptibility of the substrate material, silicone substrate structure, dip-coating parameters (temperature, dip-coating speed, dwell time in the solution, and the like), and other such parameters can be used to create highly homogeneous and/or tailored polyphosphazene coatings with the desired thickness and morphology on the specific substrate. A variety of solvents are suitable for the preparation of the polyphosphazene solution including, for example, polar aprotic solvents. In another aspect, polar protic solvents that show some solubility in or miscibility with water will also work well. For example, suitable solvents include, but are not limited to, ethyl acetate, propyl acetate, butyl acetate, pentyl acetate, hexyl acetate, heptyl acetate, octyl acetate, acetone, methylethylketone, methylpropylketone, methylisobutylketone, tetrahydrofuran, cyclohexanone, diglyme, t-butyl methyl ether, dimethyl ether, hexafluorobenzene, tetramethyl urea, tetramethyl guanidine, dimethyl acetamide, and the like, including any combinations thereof. Mixtures of these solvents can be used, or any solvent can be supplemented with the addition of other solvents or nonsolvents, such as ethane, propane, butane, pentane, hexane, heptane, toluene, benzene, xylene(s), mesitylene, diethyl ether, water and the like. Further, other components can be added to the polyphosphazene solution, examples of which include, but are not limited to, co-solvents to adjust solubility, surfactants, adhesion agents, and the like, including any combination thereof.

In another aspect, alternatively, the polyphosphazene of the present invention can be applied to the silicone by spraying the polyphosphazene onto the silicone. For example, the polyphosphazene can be deposited on the substrate by a spray coating procedure. This method is especially suited for coating irregularly shaped articles. A solution of polyphosphazene in an organic solvent can be nebulized through a pneumatic nozzle employing an inert carrier gas at a specific pressure for breaking up the liquid feed. Alternatively, the nozzle can be a minimal pressure or a pressure-less ultrasonic type, generating a mist by breaking up the solution using ultrasonic agitation. The generated solution nebulas are targeted at the substrate to be coated and produce a conformal coating on the substrate of varying thickness depending on the exact conditions of the procedure. In yet another aspect, a supercritical solution of polyphosphazene in suitable solvents, such as carbon dioxide or dimethyl ether is created at a specific set of temperature and pressure parameters and spray coated onto the substrates in question.

A further aspect of this invention provides that the polyphosphazene can be co-extruded with the silicone during the manufacturing process for the silicone whereby the newly manufactured silicone is coated with the polyphosphazene. Alternatively, the polyphosphazene can be spin-coated onto the silicone. The spin-coating method is especially suited for forming very thin, homogeneous films on flat surfaces, where solutions of polyphosphazene polymers in suitable organic solvents can be spin-cast on the substrates in question. Solvent evaporation rates, concentration, type of solvent, the polyphosphazene concentration regime, and spin-coating parameters (temperature, spinning speed, and the like), and so forth can be used to create highly homogeneous and conformal polyphosphazene coatings with specified thickness and morphology on the silicone-containing substrate.

In still another aspect, a further procedure for coating the silicone with the polyphosphazene of the present invention is to electro-spin the polyphosphazene onto the silicone. Thus, any number of methods may be used, including spraying, dip-coating, electro-spraying, spin-coating, electro-spinning, and the like. Yet another procedure for coating the silicone with the polyphosphazene is to precipitate the polyphosphazene onto the silicone. One example of such a procedure is to volatilize the polyphosphazene in the presence of a gas atmosphere, either a reactive gas or an inert gas, in a vapor deposition procedure. Alternatively, the polyphosphazene can be applied to the silicone in a reduced gas atmosphere.

In yet another aspect, the silicone-containing substrate can be coated with a polyphosphazene of the present invention by pre-forming a polyphosphazene membrane and then applying the membrane to the silicone-containing substrate, or contacting the polyphosphazene with the silicone-containing substrate. The membrane can be applied using adhesion promoters as described herein, or alternatively by solvent welding the membrane to the substrate wherein the solvent modifies the surface of the substrate in a manner that the membrane will bind to the substrate. Examples of forming a membrane of a polyphosphazene are provided in U.S. Pat. No. 7,265,199, the entirety of which is hereby incorporated by reference. While not bound by theory, it is believed that a semi-interpenetrating network between the two components is formed. However, this invention encompasses any combination of silicone and polyphosphazene, including a pre-formed polyphosphazene membrane is applied to a silicone-containing substrate, regardless of any mechanism by which the polyphosphazene and silicone might interact.

In yet another aspect, procedures such as those disclosed herein can be carried out one or multiple times. For example, a polyphosphazene layer can be applied to a silicone substrate one or multiple times. When multiple applications are employed, the thickness of the polyphosphazene coating can be adjusted or manipulated. In one embodiment, the polyphosphazene coating is substantially one polymer monolayer in thickness, that is, the coating corresponds to the dimension of the radius of gyration of a single polymer chain. In another embodiment, the polyphosphazene coating is between one monolayer and about 1 μm in thickness. In another embodiment, the polyphosphazene coating thickness is from about one monolayer to about 2 μm, or from about one monolayer to about 3 μm, or from about one monolayer to about 4 μm, or from about one monolayer to about 5 μm, or from about one monolayer to about 10 μm, or from about one monolayer to about 20 μm, or from about one monolayer to about 30 μm, or from about one monolayer to about 40 μm, or from about one monolayer to about 50 μm, or from about one monolayer to about 75 μm, or from about one monolayer to about 100 μm, or from about one monolayer to about 150 μm, or from about one monolayer to about 200 μm, or from one monolayer to about 300 μm, or from one monolayer to about 350 μm. One skilled in the art will appreciate the thickness of the polyphosphazene can be varied and can depend on the specific application or intent of use of the device or medical device.

In a further aspect, the polyphosphazene of the present invention can be added to the silicone by blending the polyphosphazene with the silicone. For example, the polyphosphazene can be blended with the silicone during the manufacturing process for the silicone. For example, after silicone elastomers are polymerized but prior to crosslinking, polyphosphazenes can be added to the silicone, and the mixture subsequently can be subjected to one or more various crosslinking procedures or reactions. For example, crosslinking procedures include radical crosslinking, condensation crosslinking, addition crosslinking, and the like. Alternatively, the silicone elastomers can be crosslinked and then the polyphosphazene added prior to curing procedures, such that the silicone and the polyphosphazene are blended in a manner, concentration, or degree as desired. In still a further aspect, the polyphosphazene can be added to the silicone during an injection molding process, such that during the molding process the silicone and the polyphosphazene are blended as desired. Depending on the processing parameters used, for example, thermal crosslinking or curing at ambient or elevated temperatures, substantially homogeneous combinations of silicone and the polyphosphazene can be obtained, as understood by one of ordinary skill.

During the various silicone manufacturing processes that are possible, the polyphosphazene can be added, for example in a blending process, with the silicone as required to achieve a desired final or pre-selected concentration of the polyphosphazene. For example, during a silicone synthesis procedure, the polyphosphazene can be added to the silicone in a specific amount, specific concentration, or at a specific rate, such that a final pre-selected concentration of the polyphosphazene relative to the composition comprising a silicone and a polyphosphazene is achieved.

In a further aspect, the polyphosphazene of the present invention alternatively can be added to the silicone by grafting the polyphosphazene to the silicone. One procedure for grafting the polyphosphazene to the silicone comprises co-extruding the two components, whereby the silicone is partially cured and the polyphosphazene is applied to one or more surfaces of the partially cured silicone, such that those two components mix or graft themselves together in a stable configuration. This grafting method can be applied to one surface of the silicone or more than one surface of the silicone. For example, silicone-based tubing can be co-extruded with a polyphosphazene of the present invention, such that only the inner or the outer surface of the tubing is grafted with the polyphosphazene. Alternatively, both the outer surface and the inner surface of the tubing can be grafted with the polyphosphazene. In another aspect, the crosslinked and polymerized silicone can be partially solubilized on one or more surfaces and a polyphosphazene added to the partially solubilized surface. Once applied, these materials then can be allowed to re-cure, such that the polyphosphazene is grafted to one or more surfaces of the silicone.

In still a further aspect, several steps or laboratory procedures typically are used when the polyphosphazene of the present invention is combined with the silicone. Depending on the nature of the substrate and the intended application, a substrate first may be cleaned if desired, for example, by ultrasonication or by immersing the substrate material into various liquid chemical cleaning baths, solutions, or reagents, followed by rinsing with an appropriate solvent based on the particular cleaning bath. Examples of cleaning reagents include, but are not limited to, oxidizing, acidic, or alkaline etching solutions. After several such cleaning steps, substrates then may be immersed in solutions containing a surface reactive adhesion promoter, for a time period sufficient to afford the desired mono- or multilayers of the adhesion promoter on the substrate. Typically, excess, unreacted reagents may be removed by further cleaning, which can be followed by a final drying step.

In another aspect, physical grafting of a polyphosphazene film onto a substrate typically is carried out by preparing the substrate by chemically grafting an adhesion promoting layer onto the surface prior to coating the surface with a polyphosphazene film of the present invention. In one aspect, to facilitate the chemical bonding of an adhesion or tie layer to the substrate, the substrate surface may be enriched with hydroxyl groups which may serve as anchoring sites for an adhesion promoter. For example, silicone substrates may be plasma activated to create a suitable reactive, hydroxylated surface, or alternatively, silicone substrates may be treated with acidic, basic, or oxidizing chemical reagents. While not intending to be bound by theory, it is thought that among other things, this procedure serves to create the desired attractive interfacial forces between the substrate and the polyphosphazene film, which helps prevent delamination of the polymer film by adhesive failure. This procedure can also serve to adjust the surface energies of substrate and the polyphosphazene coating solution, to prevent the dewetting of the solution during coating, and thereby deposit a homogeneously structured film.

For example, silicone can be submerged in a dilute solution of potassium or sodium hydroxide, thereafter washed and subsequently treated with an adhesion promoter. For example, the silicone can be submerged in a 5.7% (weight-to-volume) base solution for a period of time which can be adjusted based on the concentration of the base, the type of silicone, the degree of crosslinking of the silicone, the temperature, and so forth, thereafter washed, and then, after deposition of the adhesion promoter, contacted with a polyphosphazene. Using a 5.7% (weight-to-volume) base solution, a typical immersion time is from about 1 to about 10 minutes for many silicones.

In one aspect of this invention, the adhesion promoters may be utilized in the following manner. In general terms, for example, the interface between a substrate and the polyphosphazene polymer of the present invention may include an adhesion promoter or linker. For example, in one aspect, the adhesion promoter can comprise an acid component and an amine component. The acid component and the amine component can be situated in different substances, materials, or molecules, or within a single substance, material, or molecule. In this aspect, for example, the orientation of the adhesion promoter components relative to the substrate and the phosphazene polymer of the present invention can be represented generally in the following way:

Substrate-Acid Component-Amine Component-Phosphazene Polymer.

In this aspect, the acid component can comprise any moiety that provides an acid functionality and can be selected from, for example, acids, esters thereof partial esters thereof, or acid halides, which form hydroxyl (OH⁻) groups upon hydrolysis with water. Examples of materials that provide acid components include, but are not limited to, carboxylic acids, phosphoric or phosphonic acid derivatives, sulfuric or sulfonic acid derivatives, orthosilic acid derivatives, boronic acid derivatives, titanic acid derivatives, and all other known species, compounds, compositions, mixtures, or moieties that are known to form OH⁻ groups upon hydrolysis with water. In this aspect, the linkage with the amine (or amidine) component may be established by, for example, a typical amide linkage which results from the reaction of the acid component with the free amine and subsequent dehydration. In another aspect, the amide linkage also may be established with the elimination of halide groups instead of hydroxyl, when the acid component comprises an acid halide. While not intending to be bound by theory, the substrate-acid component linkage itself may established by ether formation or hydrogen bonding, or by any method by which the acid moiety or component may interact effectively with the substrate. In another aspect, for example, amino acids are useful as adhesion promoters and provide prototypical examples of molecules in which the acid component and the amine component are situated within a single molecule.

In one aspect of this invention, aminoalkyltrialkoxysilanes such as aminopropyltrialkoxysilanes work well as adhesion promoters when used in combination with polyphosphazenes and silicones, examples of which include compounds according to formulas II and III, illustrated here. 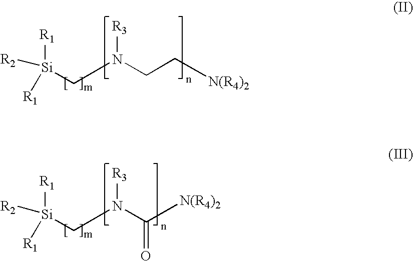
In formulas II and III, R₁ can be selected from -Oalkyl, -Oalkyl ester, or alkyl; R₂ can be selected from -Oalkyl, R₃ can be selected from H or alkyl; and R₄ can be selected from H or alkyl, wherein alkyl is defined herein, and wherein at least one of R₁ or R₂ comprises a hydrolyzable -Oalkyl group. Because at least one of R₁ or R₂ comprises a hydrolyzable group, a hydrolysis reaction can occur to form a covalent surface grafting. Further regarding formulas II and III, m can be an integer from 0 to about 20, and m is typically an integer from 2 to 12, with m being 3 being preferred. In addition, n can be an integer from 0 to 4, with m typically being selected from 1 or 2. For example, in one aspect, R₃ and R₄ can both be H, or in another aspect, R₃ and R₄ can both be CH₃, wherein m is 3 and n is either 1 or 2. While not intending to be bound by theory, it is believed that pendant groups of the siloxane adhesion promoter that have a positive dipole or quadrupole moment, whether temporary or permanent, create a favorable interaction with the negatively polarized fluorinated pendant groups of the polyphosphazene, including fluorinated alkoxide groups such as trifluoroethoxy. For example, pendant groups such as dimethylacetamido, trimethylureido, pentafluorophenyl, quaternary amines, ternary, secondary, primary amines and alkylated amides and the like, exhibit favorable adhesion.

In another aspect of this invention, an exemplary compound with a pentafluorophenyl pendant group can include the following compound of formula IV, which exhibits favorable silanole end groups.

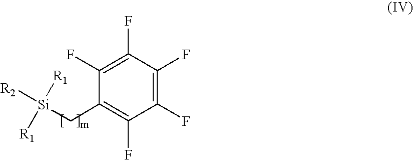

A comparison of the respective hydrolysis rates for the analogous -Oalkyl series of adhesion promoters that differ only by R₁ and R₂, wherein R₁ and R₂ are selected from OMe, OEt, or OPr, reveals a decreasing hydrolysis rate as one progresses from OMe to OPr. For example, an (OMCe)₃ terminated silane will hydrolyze 70 times faster than an (OEt)₃ endcapped silane in acidified aqueous methanol. Therefore the choice of silane end groups can be adapted to meet desired reaction times. Unless slower reaction times are required, (OMe)₃ substituted silanes are typically used.

In another aspect, for control of elastic modulus of the resulting siloxane oligomers and polymers, the crosslinking functionality can be reduced from 3 to 1 by replacing -Oalkyl with alkyl at the siloxane terminus. For example, R₁ selected from methyl may be preferred for a siloxane adhesion-promoting multilayer with increased flexibility.

A further aspect of this invention is provided by additional silane adhesion promoters, that are well-suited for a gas-phase deposition processes, examples of which are provided below as formulas V and VI.

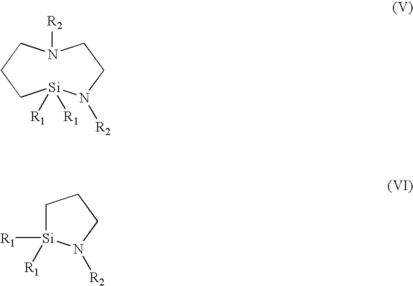

For example, in formulas V and VI, R₁ can be selected from -Oalkyl or alkyl; and R₂ can be selected from H or alkyl. Adhesion promoters of formulas V and VI, are suited for both liquid phase and gas phase silane deposition methods, regardless of whether the environment is aqueous or anhydrous. Thus, in one aspect, these adhesion promoters do not need to hydrolyze before being able to react with a hydroxyl rich surface. For example, and while not intending to be bound by theory, formulas V or VI may initiate a ring-opening sequence by reacting with surface bound hydroxyl groups immediately on contact to yield the open-chain variants. Further, reactions rates of the adhesion promoters are convenient. As described herein, such surface modifications may be performed in liquid phase, using etchants, oxidizing solutions, volatile solvents and other reactive species. Moreover, this method employing the adhesion promoters disclosed herein affords a homogeneous and smooth deposition of the adhesion promoter, and film thicknesses will depend on the concentration and deposition time of the adhesion promoter.

In Table 2, examples of each of these individual components, namely substrate, adhesion promoter as described by acid component and amine component, and the polyphosphazene are illustrated. While examples of complete acidic components are provided in Table 2, only examples of the amine portion or a molecule or composition that can constitute the amine component are illustrated, where R can be an alkyl, aryl, substituted alkyl, and the like, as understood by one of ordinary skill. Any individual component is interchangeable with any other individual component from within the same modular component type (column). Taken together, Table 2 provides a modular component “library” for substrates, acid components, amine components, and polyphosphazenes.

TABLE 2
Example of a Modular Component Library for Substrates,
Adhesion Promoters, and Polyphosphazene.

| | | | Polyphospfiazene |
|---|---|---|---|
| | | | (Formula I) |
| | | | R¹ to R⁶ independently |
| Substrates | Acidic Component | Amine Component | selected from |
| Glass | (RO)₄₋ₙSi(OH)ₙ | —NHC(NH₂)(NH) | OCH₂CF₃ |
| Metals | (RO)₄₋ₙP(OH)ₙ | —NHCOR | OCH₂CH₂CF₃ |
| Silicones | (RO)₄₋ₙSi(OH)ₙ | —NHCONH₂ | OCH₂CH₂CH₃ |
| Other Polymers | (RO)₄₋ₙTi(OH)ₙ | —NHCONHR | OCH₂CF₂CF₃ |
| | (RO)₃₋ₙB(OH)ₙ | —NHR | OCF₃ |
| | | —NH₂ | |

—HOOC—CHNHR— (Amino acids)

In this aspect, for example, this method can be used when combining alkoxysilanes containing one or more haloalkyl groups, and depositing tetramethyl-guanidine or polyethyleneimine. Further, while not intending to be bound by theory, when metals are used as substrates, amino acids such as the above mentioned can be deposited directly due to metal carboxylate formation.

In a further aspect, strong chemical interactions can be employed in the adhesion promoter interactions, for example, by chemical grafting methods, and the like. For example, dialcohol side chains may be used as part of the adhesion promoter, in which case it may be possible to connect the adhesion layer to the polymer layer by forming ether bonds with the polymer side groups. This aspect would also permit fusing the ends of side chains together, instead of simply pairing them up in the typical fashion. For example, this technique is possible by using a monoprotected alcohol functional groups during substitution, in which case a polyphosphazene as a copolymer that contains a small amount of these functional side groups is obtained. In this case, the protecting groups may be moisture labile.

Techniques such as these for grafting compounds to a silicone have been described, and the invention disclosed herein is not limited to those procedures. Other examples of surface preparation of silicones prior to grafting the silicone to other compounds can be found, for example, in U.S. Pat. No. 5,494,756.

While not intending to be bound by theory, in another aspect, for example, suitable combinations of a silicone and a polyphosphazene include copolymers thereof such as random copolymers, alternating copolymers, block copolymers, graft copolymers, other copolymers, interpenetrating networks between the silicone-containing substrate and the polyphosphazene, or blends of these materials. In one aspect, for example, using the abbreviation “A” to refer to a polyphosphazene [—R^{x}₂P═N—] moiety (wherein x is an integer from 1 to 6, according to formula I having a [—P═N—] backbone, and using the abbreviation “B” to refer to a silicone [—R₂Si—O—] moiety (wherein each R is independently a silicone substituent such as those disclosed herein) having a [—Si—O—] backbone, some of the polymers, structural motifs, and silicone-polyphosphazene combinations that are encompassed by this invention can be depicted as follows.

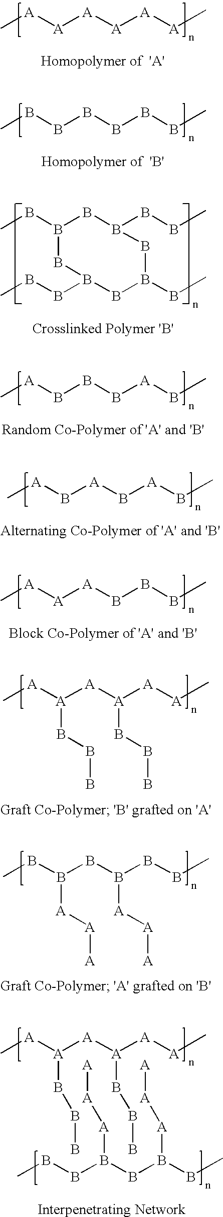

Again, while not intending to be bound by theory, in addition to the silicone-polyphosphazene backbone-to-backbone connectivities in the illustrations above, other aspects of this invention includes silicone-polyphosphazene combinations characterized by the following structures: one or more side group(s) of one polymer connecting to one or more backbone units of the other polymer; connections of one or more side group(s) of one polymer to one or more side group(s) of the other polymer; and/or all possible permutations thereof. Furthermore, these connectivities are not limited to two polymers forming a copolymer, but also can include a third or even additional polymers, or a suitable linking moiety participating in the bond formation between the polymers, including between the backbone or side groups. Therefore, this aspect also encompasses tie layers or adhesion promoters such as ethyleneimines, aminosilanes, and the like as described herein.

A blend of polymers can be described as a mixture of silicone and polyphosphazene polymers, commonly formed by using a suitable cosolvent for each polymer, or using a melt. The formation of a homogeneous or intergradient blend can be achieved in addition to the formation of a heterogeneous blend with more than one interphase. All ratios of silicone and polyphosphazene polymers in a blend are encompassed by this invention.

Again, while not bound by theory, it is thought that an interpenetrating network can be understood in terms of polymer chains (backbone units with side groups) diffusing from one polymer into the other, and interacting with polymer chains of the other in order to create a proper adhesion between the different polymers. In this aspect, the term semi-interpenetrating network is often used, as one polymer (for example, the silicone-containing polymer) comprises a crosslinked polymer chain, while the other polymer (the polyphosphazene) can be non-crosslinked and is diffusing into the other polymer. A semi-interpenetrating network can differ from the interpenetrating network by one or more polymer(s) being crosslinked and forming a stable network matrix while the other polymer is non-crosslinked. In a true interpenetrating network, which is another aspect of this invention, both polymers can be crosslinked.

Several synthetic strategies can be used to form the combinations or copolymers disclosed above. In this aspect, for example, copolymers can be formed by copolymerizing a suitable mixture of monomeric precursors or small, low molecular weight oligomers of a silicone and a polyphosphazene at the same time or at similar times. By attaching these monomer/precursor units of one polymer to the other polymer and then subsequently polymerizing these monomer units while being “grafted” on the backbone of the other polymer, a stable copolymer can be formed. In this context, this can be effected by copolymerizing suitable phosphazene precursors with suitable siloxane precursors or a silicone polymer chain. In this example, this method would provide a copolymer of A grafted on B, wherein polyphosphazene chains (and/or their precursors) are grafted on the backbone of a siloxane.

This type of grafting process can also involve a stepwise increase in molecular weight of the grafted polyphosphazene side chains in relation to the distance of the silicone polymer phase to the polyphosphazene phase. A gradual shift in molecular weight will increase the diffusion of the polyphosphazene polymer into the silicone polymer phase while allowing a gradual transition in surface energy, resulting in an even stronger adhesion between the two polymers.

In another aspect, this type of grafting could also be achieved by using polyphosphazene polymers that contain siloxane anchor groups at the terminal positions of the polymer. Due to having hydrolytically labile alkoxy substituents, these would combine with the silicone polymer during curing.

In a further aspect, the copolymer can be formed by grafting reactive silicone groups to the polyphosphazene polymer backbone with suitable reactive short-chain siloxane side groups. For example, a polyphosphazene polymer containing a suitable number of siloxane “anchor” groups can be synthesized that can undergo curing reactions similarly to that of standard silicones. Due to the hydrolytic nature of the—[NP—(OSiR¹R²R³)₂]ₙ—bonds, it would be preferable to use bulky substituents (R¹, R², and/or R³) on the silicon atom to afford steric protection to the polyphosphazene PN polymer backbone, and stabilize these moieties from hydrolysis, while at the same time providing a reactive substituent (at least one of R¹, R², and/or R³), that allows convenient hydrolysis and thus crosslinking to an existing siloxane network.

In this aspect, the following structure is one example of a suitable siloxane anchor group connected to, or inserted in, a polyphosphazene backbone:

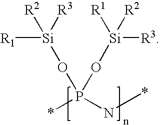

In this example, the following chemical substitution reactions depict reaction scenarios which can afford a grafted polyphosphazene siloxane copolymer. 1) The reaction of a polyphosphazene precursor such as a polychlorophosphazene or polyalkoxyphosphazene, with a metallated silanol species, with elimination of the metal halide or metal alkoxide, can afford —[NP(OSiR¹R²R³)₂]— moieties. Reagents that can be used to form a metallated silanol can include Grignard reagents, organolithium reagents, organocopper reagents, organozinc reagents, and the like. Thus, metallation of the silanol HOSiR¹R²R³ will form a metal silanolate (Mⱼ(OSiR¹R²R³)ₖ (wherein j and k depend on the identity of the metal ion) that is sufficiently reactive towards a halo-polyphosphazene or a phosphazene with labile alkoxy substituents. Metals can include, but are not limited to group 1, 2, 11, 12, 13, and 14 metals, with a preference for lithium, sodium, magnesium, aluminium, zinc, tin, or copper. 2) The reaction of a polyphosphazene precursor such as a polychlorophosphazene with a suitable amino-(organo)silane or amino-(organo)siloxane reagent, which will form the desired polyphosphazene-siloxane copolymers, with the formation of hydrochloric acid or any stable leaving group. In this later case this reaction optionally can be performed in the presence of a base.

In further aspect, additional strategies in copolymer formation include linking of side groups by suitable reagents. This could be achieved, for example, by organosilicon hydride species that is reacted with activated (organo) double bond anchor groups of a polyphosphazene polymer. Alternatively, this could be achieved, for example, by reactions at the side arms of the grafted siloxane polymer, such as fluorine displacement reactions that transfer the fluorine substituent from the fluoro-organo phosphazene side group to the silyl bearing side group.

Instead of using above-disclosed, relatively weak physical or chemical interactions such as hydrogen-bonding, stronger bonding interactions can be made by chemical grafting, when using dialcohol side chains may be used as part of the adhesion promoter. In this case, it may be possible to connect the adhesion layer to the polymer layer by forming ether bonds with the polymer side groups. This aspect would also permit fusing the ends of side chains together, instead of simply pairing them up in the typical fashion.

As disclosed herein, the formation of a stable interpenetrating network can involve a stepwise deposition of polyphosphazene layers with increasing molecular weight of the particular deposited polyphosphazene polymer in relation to the distance of the silicone polymer phase to the polyphosphazene phase. A gradual shift in molecular weight can increase the diffusion of the polyphosphazene polymer into the silicone polymer phase while allowing a gradual transition in surface energy, thereby increasing the adhesive forces between the components.

Further, the initial bonding of a primary polyphosphazene layer to a silicone can involve deposition of suitable precursors as described previously, with a subsequent thermal, radiation-induced, or plasma-induced polymerization, crosslinking reaction of the polyphosphazene or precursors thereof described previously, interdiffused within the silicone domain.

As provided herein, the disclosed devices and methods are not limited as to the exact disposition of the silicone and polyphosphazene components, and descriptions have been used such as the silicone can be coated (or layered) with, reacted with, blended (or mixed) with, grafted to, bonded to, crosslinked with, copolymerized with, coated and/or reacted with an intermediate layer that is coated and/or reacted with, or combined with in any manner with the polyphosphazene. Therefore, a polyphosphazene combined with or added to a silicone can be used to describe copolymers of these two molecules whereby one chemical moiety has been added to the other chemical moiety by bonding the two polymers together. The phrase “silicone added to a polyphosphazene” or “polyphosphazene added to a silicone” or variations on these descriptions include silicones that contain polyphosphazene side chains, or in other words, these polymers can be formed from the bonding or incorporation of polyphosphazene side chains onto or into the silicone. This bonding can be either a covalent bonding or an ionic bonding. In this aspect of adding a polyphosphazene to a silicone, the polyphosphazene can be added to the silicone in a manner whereby the thickness of the polyphosphazene is controlled and the type of chemical bonding between the polyphosphazene and the silicone is controlled by the choice of reagents or precursors, as disclosed herein.

One skilled in the art will recognize that, in addition to terms commonly used in this disclosure to describe the interaction of the silicone and polyphosphazene of the present invention, such as coating, blending, grafting, bonding, and the like, additional terms can be used to describe the various combinations of the silicone and the polyphosphazene components encompassed by this invention. In this aspect, for example, terms such as adhere, stick, glue, fix, join, bind, attach, cement, link, affix, meld, weld, fasten, fuse, amalgamate, append, affix, intermix, admix, mix, unite, integrate, merge, and combine are examples of terms which can be used rather than the terms used herein to describe adding a polyphosphazene to a silicone.

The processing techniques designed to bring about the described intrinsically or extrinsically structured polymeric composite articles achieve their tightly interconnected networks through either physical or chemical interaction or both. In one aspect, the interfacial contact area between the different polymer phases can be maximized during the bonding procedure in order to enhance the adhesion interaction between them.

Adhesion Promoters, Tie Layers, and Pre-Treatments. For enhanced adhesion between the polymeric phases, surface energy respective cohesive energy density can be matched, so that during the adding or combination process, the polyphosphazene polymer can be applied to the silicone elastomeric substrate to achieve an even and conformal contact. The surfaces of polymers such as PDMS, Silastic® and other similar silicone elastomers are usually hydrophobic, which means that they have a low surface energy and thus are not very easily coated with hydrophilic compounds or compositions. Another feature of low surface energy polymers is that these substrates can become electrostatically-charged and therefore can readily collect atmospheric dust particles. In order to achieve a good cleanliness, wettability, and improved adhesion of the polymer substrates to be coated, these substrates can be pre-treated using various techniques to “activate” their surface. Such activating techniques are aimed at increasing the substrate's polarity as well as raising the surface energy to increase the adhesive power, wettability, and non-electrostatic and non-soiling characteristics.

Thus, the methods disclosed herein are applicable to Silastic®, which itself is a silicone, containing a number of dimethylvinyl terminated dimethylsiloxanes, which can be used to form copolymers with Latex (which itself is a polymer based on isoprene Units). Isoprene and dimethylvinyl groups are illustrated below. Curing of the copolymerized Latex and Silastic® materials may be achieved by either Platinum catalysts (Addition type) or Peroxide curing (Heat). Such processes are applicable to a molding process, for which heat and peroxide curing are useful, in which toluene is a common solvent and benzoyl peroxide is a useful curing agent.

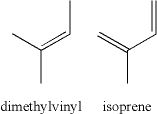

Numerous examples of substrate pre-treatments have been used that can provide sufficient surface activation of the polymeric surfaces. In this aspect, typical procedures include, but are not limited to, wet chemical treatments with aggressive chemical baths containing acidic, basic, or oxidative solutions. Such procedures can be used in the present invention to aid in the adhesion and/or bonding of the polyphosphazene to the silicone or silicone-containing substrate.

In this aspect, for example, the polymeric substrates can be swelled in (halo-) organic solvents and then treated with an oxidizing solution containing chromic-sulfuric acid, nitric acid, (hydrogen-) peroxides, peroxodisulfates, Caro's acid (persulfuric acid, SO₂(OH)(OOH)), ozone, and the like. Other pre-treatment procedures include the wet chemical treatment of the polymeric substrates with bromine saturated water or the treatment with alkaline solutions based on alkali or alkali earth hydroxides. Still other treatments include the reaction of polyimide surfaces with hydrofluoric acid or sodium itself to bring about the desired changes in surface energy.

Other aspects of surface treatment techniques include, for example, exposing the polymeric substrate to flame pyrolysis, fluorination, actinic exposure to x-rays or other radiation, positive or negative ionizing and e-beam irradiation, corona discharge, or plasma processing to bring about the desired changes in surface energy. The latter two techniques have been widely used for the surface treatment of polymeric materials to be coated, and are briefly explained as follows.

Corona discharge usually is effected by exposing polymeric substrates to a direct current-generated, atmospheric corona (spark) discharge, creating highly reactive ozone from environmentally present air, and then reacting the upper surface of the polymeric substrate with the ozone creating an oxidized, chemically reactive, high surface energy polymer suitable for further bonding applications.

Other plasma processing techniques involve the treatment of polymeric substrates with an AC-, DC-, or microwave-generated plasma of varying power (usually several hundred up to a few thousand Watts) in either an atmospheric or low-pressure environment at room- or slightly elevated temperature, with inorganic and organic gases. Examples of inorganic and organic gases include, but are not limited to, argon, helium, nitrogen, hydrogen, nitrous oxide, oxygen, air, hydrogen chloride, fluorine, bromine, chlorine, carbon monoxide, carbon dioxide, ammonia, methane, alkanes, aromatic compounds, haloalkanes and aromatic compounds, and similar compounds either alone or in suitable combinations. Such plasma process can effect the desired changes in surface energy and chemical functionality.

As this aspect applies to silicone-containing substrates, it is relatively easy to monitor the influence of a plasma activation treatment on a silicone-containing substrate to verify changes of the surface energy. For example, one such method is to measure contact angles of substrates prior to and after plasma treatment. A native plastic substrate typically displays high contact angles due to the hydrophobic nature of the material. After plasma activation, for example following plasma activation in a nitrogen/oxygen atmosphere, substrate surfaces are rendered hydrophilic due to the generation of hydroxy-groups on the surface. Contact angles therefore will be decreased considerably after plasma activation.

The plasma activation process is quite gentle to substrates and can be repeated several times if necessary. The amount of time needed for effective surface treatment can be decreased until the contact angle of the substrate stays constant. The risk of substrate etching occurs only after increased periods of continuous plasma treatment, usually more than about 15 minutes or so. The treated substrate surface can typically remain active for approximately ten minutes to several hours, but this time can vary, based on the individual treatment, the conditions under which the activated surface is maintained, or any reactive species the activated surface can come into contact with following activation.

Once the substrates in question have been sufficiently cleaned and activated by one of the aforementioned methods or by similar techniques, the substrates can be subjected to further treatments to bring about the desired surface functionality necessary for creating a chemically- or physically-reactive surface or layer for the polyphosphazene to be reacted with, blended, grafted or otherwise combined with the silicone-containing substrate. As disclosed above for the wet chemical methods and the dry techniques, the polymeric substrates can be contacted with surface modification agents, either in a liquid or gaseous state.

For imparting the desired surface functionality in plasma and corona discharge based techniques, gaseous oxygen for example, can be used to generate hydroxy-, carboxy-, aldehyde-, or peroxy-groups on a polymeric substrate. Ammonia can be used to impart amino- or imino-functionality to a surface. Further, hydrogen can be used to provide a hydride-functionality to a silicone surface. Therefore, as understood by one of skill in the art, the surface functionality can be tailored by selection of the reagent gas under which the plasma and corona discharge is carried out.

In the preceding aspects whereby a polyphosphazene is added to a silicone, these procedures can also be supplemented with a number of steps or reagents that can aid in the process of adding the polyphosphazene of the present invention to the silicone. In one aspect, a compound or composition can be included to the procedure of contacting or adding the silicone and the polyphosphazene to facilitate adhesion of the polyphosphazene to the silicone. For example, an adhesion promoter or a spacer can be added to the silicone surface, added to the polyphosphazene, blended into the silicone or the polyphosphazene, grafted to the silicone, or bonded to the silicone or the polyphosphazene prior to adding the polyphosphazene to the silicone.

While not intending to be bound by theory, in this aspect, the adhesion promoter can improve adhesion of the polyphosphazene to the silicone by coupling the adhesion promoter to both the silicone and to the polyphosphazene, for example, by ionic and/or covalent bonding, or by other lower energy interactions such as van der Waals or hydrogen bonding interactions, or combinations thereof. In one aspect, for example, the attachment of the polyphosphazene to a silicone-containing substrate can be enhanced by a plasma activation step of the silicone to create reactive moieties, such as hydroxylated surfaces or layers, which can bond to the adhesion promoter or the polyphosphazene.

Further to this aspect, the adhesion promoter or spacer can contain a polar end-group, examples of which include, but are not limited to, hydroxy, carboxy, carboxyl, amino, nitro groups, and the like. Further, the O-ED type end groups can also be used, wherein “O-ED” stands for an alkoxy, alkylsulfonyl, dialkyl amino, or aryloxy group, or a heterocycloalkyl or heteroaryl group with nitrogen as the heteroatom. In this case, the O-ED type end groups can be unsubstituted or substituted by, for example, halogen atoms, such as chlorine or fluorine. In this aspect, fluorine-substituted O-ED groups work well.

In yet another aspect of this disclosure, the adhesion promoter can comprise or be selected from monosilanes, oligosilanes, polysilanes, monoethylene imines, oligoethylene imines, polyethylene imines, or cyclic polyphosphazene precursors. For example, treatment of silicone and polyphosphazene surfaces can include surface adhesion promoters comprising an ethyleneimine-monomer, -oligomer, or polymer intermediate layer (tie layer), which can be reacted, grafted, or otherwise bonded to both substrate surfaces by any chemical or physical interaction. For example, a chemical interaction can be effected by a suitable crosslinking reaction that can permanently bond the intermediate (tie) layer to both the silicone and the polyphosphazene.

Crosslinking of the (poly)ethyleneimine (PEI) tie layer can be brought about a number of methods, including, but not limited to, reaction of the tie layer, the silicone and/or the polyphosphazene composite layers, or a combination thereof, using at least one reagent such as the following. Possible crosslinking reagents include, but are not limited to, an (di)aldehyde (for example, terephthaldehyde), an alkyl (di)halide (for example, ethylene dibromide), isocyanates and/or thioisocyanates (for example, 4-nitrophenyl isothiocyanate, 4-nitrophenyl isocyanate), activated double bond compounds (such as vinylic, acrylic, and/or acrylonitrilic compounds), epoxy compounds (such as epichlorohydrin or oxirane), or by forming stable amides with cyanamide, guanidine, urea, or related compounds.

Further, crosslinking can also effected by forming condensate products with carboxylic acids, carboxylic acid chlorides, carboxylic acids, carboxylic acid anhydrides, or other reactive carboxylic acid derivatives such as ethyl chloroacetate, to form stable carboxylic acid amides.

Another means of bonding a tie layer to the silicone surface involves the use of photochemically-active compounds, such as acrylic, vinylic, nitro-aromatic, fluoro-phenyl, benzophenonyl, and/or azo-compounds that crosslink spontaneously upon irradiation.

Any of these crosslinking agents can contain one, two, three or more active chemical groups to bring about the formation of a one-, two-, or three-dimensional polymeric network, in order to create proper adhesion between the polyphosphazene polymer and the silicone-containing substrate.

Other ways of chemically bonding a polyethyleneimine film on the surface of a silicone substrate include, but are not limited to, reaction of ethyleneimine monomer (“aziridine”) gas with a properly-activated silicone surface. The activated surface provides chemically reactive units that bond the monomers and initiate polymerization of the subsequent units. This activation usually involves oxidative pre-treatment methods described herein to form surface silicone hydroxyl groups.

In one aspect of this disclosure, one useful method for preparing and activating the silicone is activation of a silicone surface by plasma, and the dosing of ethyleneimine (aziridine) gas into the plasma chamber. In this procedure, a homogeneous or near homogeneous tie layer of polyethyleneimine is formed on the surface of the substrate. One advantage of this method lies in the covalent bonding of the aziridine which results by ring opening to the substrate and forming a C—O ether bond that results by nucleophile attack of the hydroxyl groups located on the silica/silicone surface. The remaining amino functionality is then available for reacting with further aziridine molecules, or available for forming a layer of positively charged amino groups that will physically attract a negatively charged polyphosphazene polymer film.

Other suitable chemical activation methods of a silicone surface in order to incorporate (poly) ethyleneimine can include, but are not limited to, the conversion of surface Si—OH (hydroxyl) groups into more reactive groups, like halide groups (F, Cl, Br, or I), especially chloride groups, by the use of a chlorinating agent such as thionyl chloride, phosphorus chloride, phosphorus oxychloride, and/or oxalyl dichloride. The reaction of a water-free, anhydrous (poly)ethyleneimine (for example, dissolved in an organic solvent or using ethyleneimine monomer gas) with this type of activated (chlorinated) silicone surface can generate a homogeneous or near homogeneous tie layer on the silicone surface.

The polyethyleneimine layer can also be bonded to the silicone by the use of an intermediate (3-aminopropyl)trimethoxysilane (APTMS) layer between the silicone and the (poly)ethyleneimine (PEI). Subsequent crosslinking can then occur between the amino-end groups of the APTMS tie layer and the amino groups of the (poly) ethyleneimine (PEI). In the case of using alkoxysilanes as adhesion promoters, one solvent of choice that is very useful is the analogous alcohol that results from the hydrolysis of the silicone precursor, which for APTMS, is methanol.

By using any of these described activation methods, a (poly)ethyleneimine film can be deposited with sufficient surface adhesion on a silicone surface or layer and subsequently with the polyphosphazene substrate.

In one aspect of this invention, physical interaction between the substrates and the tie layer can be established to aid in combining the siloxane and the polyphosphazene. By the term “physical interaction”, it is meant to include such interactions as electrostatic interactions, either electrostatic interaction alone, for example by forming ionic pairs such as ammonium carboxylates by reacting polyethyleneimines with carboxylic compounds, or by the attraction of the two oppositely charged polymeric surfaces alone.

In another aspect of this disclosure, the adhesion promoter can be an organosilicon compound, such as an amino-terminated silane, or based on aminosilane, amino-terminated alkenes, nitro-terminated alkenes, and silanes, or an alkylphosphonic acid. Concerning the various silane-based adhesion promoters, these can include ureido- and glycidyl-terminated silanes which are especially useful for bonding of epoxy resins, thiol or acroyl termini which can be employed for bonding to vinylogous and acrylate based rubbers, or other substrates disclosed herein. For fluoroelastomers, amine and perfluoro based silanes are generally preferred. Other examples of silane-based adhesion promoters include N-(2-aminoethyl)-3-aminopropyltrimethoxysilane, bis[(3-trimethoxysilyl)propyl]-ethylene diamine, and other commercially-available functional silane reagents. In one aspect, a particularly useful silane-based adhesion promoter is (3-aminopropyl)trimethoxysilane (APTMS).

In typical chemical vapour deposition and plasma polymerization techniques, previously cleaned and activated polymeric substrates can be further reacted with unsaturated, crosslinkable, monomeric, chain-forming reactant gases which under plasma conditions form highly crosslinked polymeric coatings on the substrate. For example, suitable gases include ethylene imine, allyl amine, cyanoethylene, acetylene, or other similar compounds, especially unsaturated compounds. Such plasma polymerized films and modified surfaces or layers can act as an adhesion promoting tie layer for further bonding of other polymeric films, including a polyphosphazene film.

In still a further aspect, as an alternative or an additional step to using vapour deposition and/or plasma polymerization techniques, the activated surfaces also can be subjected to a liquid treatment involving solutions of surface active agents such as mono- oligo- or polymeric anionic, non-ionic or cationic surfactants, and generally compounds that impart a positive, negative, ionic or any other specifically desired functionality to the surface. These functionalized and respectively charged substrate surfaces can act as adhesion promoting tie layers for further bonding of other polymeric films, including a polyphosphazene film.

In a further aspect of this disclosure, other reactions of the substrate that can aid in the combination of the silicone substrate with the polyphosphazene can include grafting mono-, oligo-, or polymeric moieties from solution onto the plasma activated substrate. Suitable compounds can also be coated as uncrosslinked, non-polymerized mono-, oligo-, polymeric solutions. Examples of suitable compounds include, but are not limited to (oligo-, poly-) ethylene imines, (oligo-, poly-) diallyldimethylammonium chlorides, (oligo-, poly-) ethylene oxides, (oligo-, poly-) acrylates, and (oligo-, poly-) silanes, which then can be polymerized and grafted to the substrate. This polymerization-grafting process can occur either by physically subjecting the coated substrate to heat or (positive/negative) ionizing-, actinic-, X-ray irradiation, UV-light, or chemically by employing thermal or light-curing, transition metal based peroxide-, azo-, and other typical polymerization catalysts known in the art.

In a further aspect, additional steps can be employed in combination with the activation methods and other steps disclosed above for adding a polyphosphazene of the present invention to the silicone-containing substrate. For example, the substrate can be treated with a cleaning agent, such as a chemical cleaning agent, or the substrate can be subjected to another treatment whereby contaminants on the surface or layer of the substrate are removed. These methods can comprise washing the substrate with a chemical agent such as an oxidizing agent, an acidic solution, an alkaline solution, or a reducing agent, that can possibly etch the silicone-containing substrate. A separate drying step optionally can also be employed.

In further aspects, this disclosure provides methods for making a medical device comprising a polyorganosiloxane in combination with a polyphosphazene of the present invention. This disclosure also provides methods of imparting improving properties to the medical devices by, for example, reducing cell encrustation, reducing the severity of thrombosis, or improving the anti-rejection properties of the medical device. Also provided by this disclosure are methods of imparting antibacterial and/or antithrombogenic properties to a medical device that contains a polyorganosiloxane, the method comprising adding to the polyorganosiloxane or combining with the polyorganosiloxane at least one polyphosphazene of the present invention.

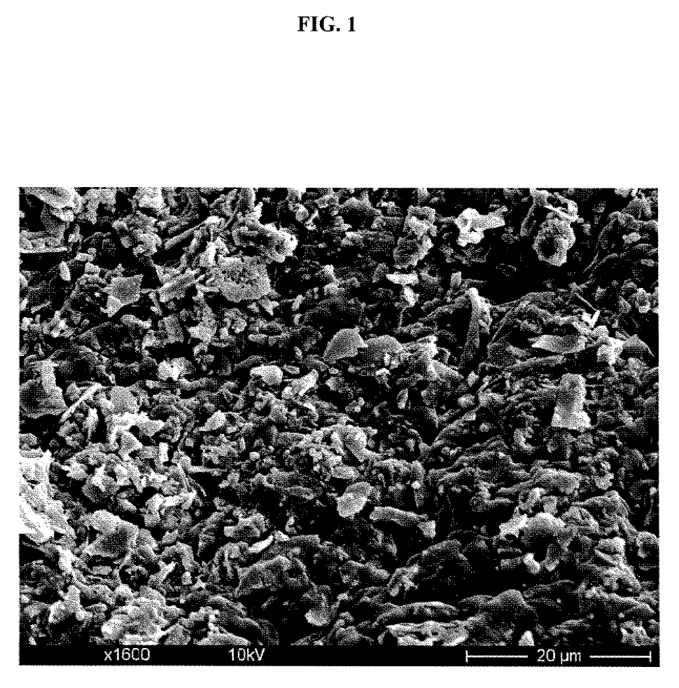

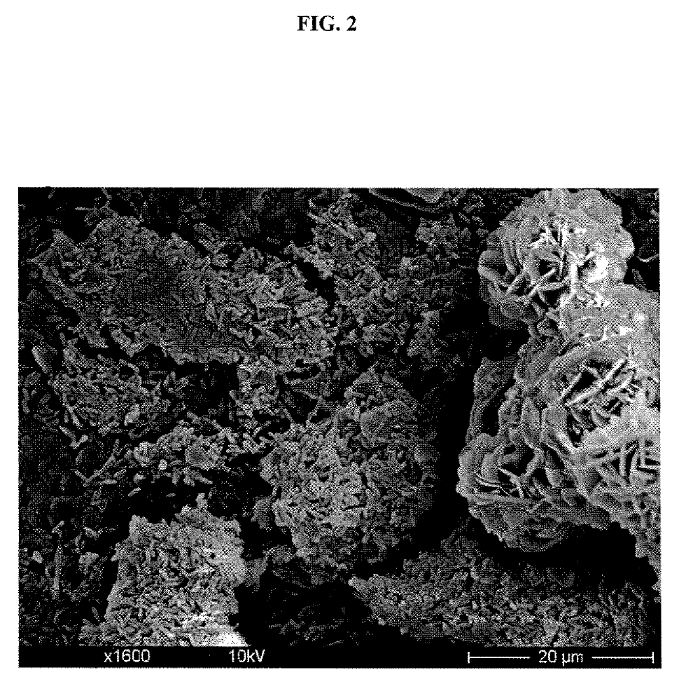

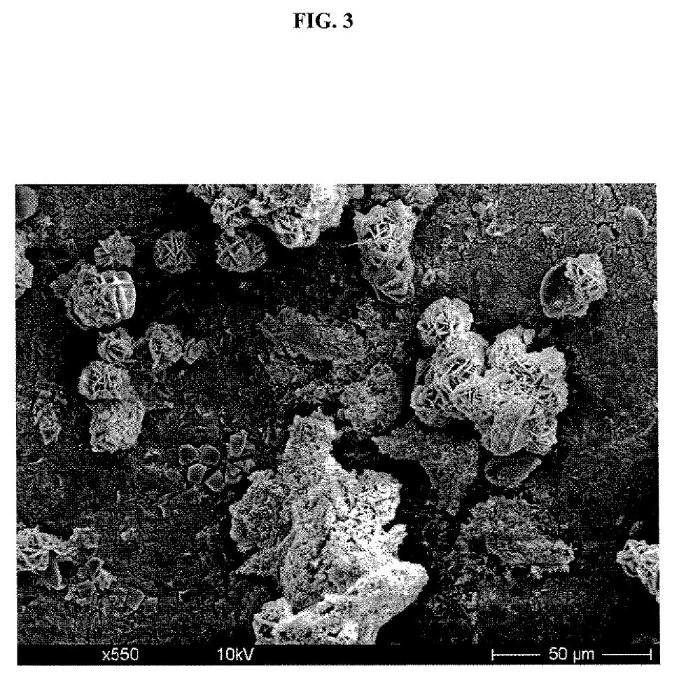

Referring to FIGS. 1 through 3, a series of scanning electron microscope (SEM) images are shown that illustrate one manner by which the present invention can impart more biocompatible properties to a device. FIGS. 1 through 3 are images of a surface of a Silastic® Foley catheter that were taken after a 3-day incubation in artificial urine containing E. coli. In FIG. 1 (1600×), the Silastic® Foley catheter was treated with poly[bis(2,2,2-trifluoroethoxy)]phosphazene according to this disclosure, and then subjected to the 3-day incubation period. In FIG. 2 (550×) and FIG. 3 (1600×), the Silastic® Foley catheter was not treated with any polyphosphazene, and then was subjected to the 3-day incubation period. As these SEM data illustrate, no significant calcification or mineralization of the polyphosphazene-treated Silastic® catheter was observed at the end of the 3-day incubation period (FIG. 1), whereas the untreated Silastic® catheters exhibited significant calcification after the 3-day incubation period (FIGS. 2 and 3). Thus, the FIGS. 2 and 3 samples clearly show more crystal formation, where the mineral deposits appear as the needle-shaped material. Therefore, in still another aspect, the present disclosure also provides a method of reducing calcification of a polyorganosiloxane-containing device that has contact with tissue or fluids of the human or animal body or organ, comprising adding a polyphosphazene to the polyorganosiloxane. As described herein, this method also is not limited as to the exact disposition of the polyorganosiloxane and polyphosphazene components, for example, the polyorganosiloxane can be coated with, blended with, mixed with, grafted to, bonded to, layered on, or combined with in any manner.

In summary, the present disclosure provides methods and devices and related inventions whereby a polyphosphazene is added to a silicone-containing device to provide the device with enhanced and superior properties relative to the device in the absence of the polyphosphazene. In particular, the silicone-polyphosphazene device has enhanced antibacterial properties, antithrombogenic properties, enhanced flow characteristics, enhanced lubricity, enhanced biocompatibility properties, enhanced resistance to degradation, and anti-rejection properties.

The present invention is further illustrated by the following examples, which are not to be construed in any way as imposing limitations upon the scope thereof. On the contrary, it is to be clearly understood that resort can be had to various other aspects, embodiments, modifications, and equivalents thereof which, after reading the description herein, can suggest themselves to one of ordinary skill in the art without departing from the spirit of the present invention or the scope of the appended claims.

It is to be understood that this invention is not limited to specific devices, substrates, types of silicone, polyphosphazenes, or other compounds used and disclosed in the invention described herein, including in the following examples. Each of these can vary. Moreover, it is also to be understood that the terminology used herein is for the purpose of describing particular aspects or embodiments and is not intended to be limiting. Should the usage or terminology used in any reference that is incorporated by reference conflict with the usage or terminology used in this disclosure, the usage and terminology of this disclosure controls.

Unless indicated otherwise, parts are reported as parts by weight, temperature is reported in degrees Centigrade, and unless otherwise specified, pressure is at or near atmospheric. An example of the preparation of a polyphosphazene of this invention is provided with the synthesis of poly[bis(trifluoroethoxy)phosphazene] (PzF) polymer, which is prepared according to U.S. Patent Application Publication No. 2003/0157142, the entirety of which is hereby incorporated by reference.

Also unless indicated otherwise, when a range of any type is disclosed or claimed, for example a range of molecular weights, layer thicknesses, concentrations, temperatures, and the like, it is intended to disclose or claim individually each possible number that such a range could reasonably encompass, including any sub-ranges encompassed therein. For example, when the Applicants disclose or claim a chemical moiety having a certain number of atoms, for example carbon atoms, Applicants' intent is to disclose or claim individually every possible number that such a range could encompass, consistent with the disclosure herein. Thus, by the disclosure that an alkyl substituent or group can have from 1 to 20 carbon atoms, Applicants intent is to recite that the alkyl group have 1, 2, 3, 4, 5, 6, 7, 8, 9, 10, 11, 12, 13, 14, 15, 16, 17, 18, 19, or 20 carbon atoms. In another example, by the disclosure that a coating is between one monolayer and about 1 μm in thickness, or from about one monolayer to about 2 μm, or from about one monolayer to about 3 μm, or from about one monolayer to about 4 μm, or from about one monolayer to about 5 μm, or from about one monolayer to about 10 μm, and the like, it is intended to include sub-ranges within this disclosure, such as, for example, from about 1 μm to about to about 5 μm in thickness, and about 3 μm to about 10 μm in thickness. Accordingly, Applicants reserve the right to proviso out or exclude any individual members of such a group, including any sub-ranges or combinations of sub-ranges within the group, that can be claimed according to a range or in any similar manner, if for any reason Applicants choose to claim less than the full measure of the disclosure, for example, to account for a reference that Applicants are unaware of at the time of the filing of the application.

### EXAMPLES

The following general information is provided regarding the molecular weights and molecular weight determinations of this disclosure. A typical polyphosphazene that was used in the devices and methods of this invention typically is in the molecular weight range of from about 10 million kg/mol to about 25 million kg/mol, which is equivalent to values of n from about 85000 to about 215000, wherein the degree of polymerization is given by the number n of repeating monomer units within the polymer.

The molecular weight measurements of the polyphosphazenes was determined by at least one of the following methods.

a) Viscosimetry. Viscometry measurements were taken in tetrahydrofuran solvent according to S. V. Vinogradova, D. R. Tur, V. A. Vasnev, “Open-chain poly(organophosphazenes). Synthesis and properties”, Russ. Chem. Rev. 1998, 67 (6), 515-534. The relative viscosities of poly[bis(trifluoroethoxy)phosphazene] solutions in tetrahydrofuran solvent were determined with a dilution series. The intrinsic viscosity was then calculated by extrapolating the reduced viscosities to zero concentration. The Molecular weight was then determined with the help of the Mark-Houwink equation.

b) Gel Permeation Chromatography. Gel permeation chropmatography (GPC), also called Size-Exclusion Chromatography, was conducted in cyclohexanone according to the method provided in T. H. Mourey, S. M. Miller, W. T. Ferrar, T. R. Molaire, Macromolecules 1989, 22, 4286-4291.

Both Viscometry measurements and GPC methods gave agreeable results within an error margin of ±2×10⁶ g/mol molecular weight. The GPC analysis show a monomodal molecular weight distribution proving the absence of oligomers with a sharp polydispersity index of less than about 1.6. Polydispersity measurements were typically in the range of about 1.2 to about 1.4.

### Example 1

### General Procedure for Plasma Cleaning and Activation

Cleaning of the substrates and creation of reactive anchoring sites for the adhesion promoter molecules is achieved by a 1-30 min plasma treatment at reduced pressure (typically, 0.01-10 mBar), employing a 70-100/0-30 (v/v) % (nitrogen or argon)/oxygen mixture as a reactant gas mixture inside a vacuum chamber. The nitrogen/oxygen plasma itself is created through an RF excitation of variable magnitude, most preferred but not limited to an AC field frequency of 13.56 MHz at a variable power of 100-300 watt. The reaction is carried out at room temperature. To avoid overheating the substrates, the RF field can be pulsed periodically to dissipate the generated heat. Adventitious carbon from ubiquitous organic matter, silicone oils and other residual contaminants stemming from the processing of the silicone elastomeric products are thereby eliminated from the substrate surface by reaction with the highly reactive plasma.

The resulting gaseous reaction products are removed by purging the chamber. The substrate surfaces are slightly roughened during the plasma processing, thereby leading to an increased interfacial contact area. The reactive oxygen plasma yields a negatively charged substrate surface enriched in hydroxyl groups, especially suited for grafting of mono-, oligo-, and/or polymeric silanes, cationically-charged surfactants, polyelectrolytes, and the like. A further advantage of plasma treatment at reduced pressure is based on the wetting characteristics obtained. For example, plasma-cleaned and -activated substrates can be homogeneously wetted by liquid modification agents, which can lead to deeper penetration and more efficient surface modification of the substrate.

### Example 2

### Process for Plasma Cleaning and Activation of a Silicone Surface

A silicone RTV compound from NuSil was coated as a 1 mm thick film onto pre-cleaned glass rods (length 60 mm; diameter 1 mm) and onto optical microscopy glass slides. The silicone compound was left to cure for 24 h at room temperature and ambient moisture. The substrates were then subjected to a pulsed 120 sec plasma treatment at ≦5 mbar in a 20/80 (v/v) % O₂/N₂ atmosphere, employing a Ilmvac PlasmaClean-4 plasma chamber. Treatment was periodically interrupted in 10 sec intervals so that total plasma treatment time amounted to about 1 minute. This procedure was repeated several times and the dynamic contact angle against water was determined after each treatment, as described below. This procedure was repeated until full surface activation took effect, as measured by when the contact angle could not be modified further even after prolonged plasma exposure. As a result, it was determined that approximately two, 1-minute (120 second total) treatments were sufficient for full surface activation. After removing of the device parts from the plasma chamber, all parts were subjected to contact angle measurements with a Dataphysics DCAT 1.2 Wilhelmy balance. The Wilhelmy balance was first calibrated with a Pt reference plate against water, after which the wetted length of each device part was determined with n-perfluorohexane, and this value was used for measuring the dynamic contact angle against water. This procedure was repeated after each consecutive coating step.

The RTV silicone compound exhibited very high water contact angles, beyond 90° in the native state. The plasma activation treatment caused a massive drop in contact angles on the silicone substrate, which indicates easier bonding of the aminosilane adhesion promoter to the silicone surface and a better spreading of the polyphosphazene coating solution. No optical deterioration was observed for any of the substrates after the plasma treatment. A second plasma treatment did not cause further drops in contact angles; therefore, a single 120 sec treatment period was sufficient for a stable surface modification.

### Example 3

### General Procedure for Optional Wet Cleaning and Activation

As an extension to the plasma cleaning and activation procedure or a stand-alone option, the silicone elastomers and any other polymeric substrate can be further subjected to a wet chemical treatment to enhance the functional density of the anchor groups suitable for bonding of the polyphosphazene specific adhesion promoter on the surface. This treatment is provided to increase adhesive strength.

The wet chemical treatment includes immersing the substrate in typically 1-10%, or 1-20%, or 1-30%, or 1-40%, or 1-50%, or 1-60%, or 1-70%, or 1-80%, or 1-90% or higher concentration solutions of aqueous alkali- or alkaline-earth containing hydroxides for periods of 1-30 min, or more. The hydroxide solutions can contain organic swelling solvents or agents for the silicone elastomeric substrates to achieve a deeper penetration of the hydroxide solution into the polymeric substrate. In this aspect, for example, the swelling solvents can be selected from alcohols or organic amines. For example, swelling agents can be selected from methanol, ethanol, isopropanol, ethylene glycol, ethanolamine, ethylene diamine, diisopropylamine, or other typical swelling reagents known to the art, or any combination thereof. Thus these swelling agents can be present in the aqueous hydroxide solution in any concentration, as the solubility of the chosen hydroxide compound permits. In one embodiment, a 5 (w/v) % aqueous KOH solution in a 7:3 (v/v) isopropanol/water mixture is used.

After the wet chemical treatment, the substrate is rinsed with deionised water for extended periods of time until all traces of alkaline are removed. The rinsing medium optionally can contain EDTA or acetic acid in suitable amounts for neutralization and simultaneous complexation of metal ions which can interfere with the subsequent processes. A final rinse with water and drying of the sample substrates either at elevated temperatures or under vacuum also can be employed, with or without this optional cleaning and activation procedure.

### Example 4

### Process for Wet Chemical Treatment

In order for plasma cleaning and activation effects to be evaluated, the surface charge and hydroxyl group density on activated 100% all silicone catheters were examined, using the positively-charged fluorescence dye, Pyronin G.

A 5 (w/v) % aqueous KOH solution in a 7:3 (v/v) isopropanol/water mixture was prepared. Plasma-treated 100% silicone tubing substrates were immersed and maintained in this solution for 15 min, after which time they where neutralized by submerging the tubing substrates into a 10 mM HOAc solution for 30 min. Following the neutralization step, the samples were triple-rinsed with deionized water. These tubing samples were then dried in a convection oven at about 60° C. for about 1 h.

Following the wet treatment process, the samples were immersed for about 120 min in a 250 mg/L Pyronin G solution prepared in a 0.1 M phosphate buffered saline (PBS) solution, after which time the samples were withdrawn, extensively rinsed with deionized water, and air-dried. The samples were then evaluated using an optical microscope at 0.65× magnification in transmission illumination.

The results of this evaluation demonstrate that for surface hydroxylation of silicone elastomers, a plasma treatment followed by immersion into an alkaline KOH solution (KOH 5 (m/v) %, 3:7 (v/v) isopropanol:water) yields an excellent negative surface charge for covalent bonding of silane adhesion promoters.

### Example 5

### General Procedure for Surface Modification of Silicone Elastomers with Adhesion Promoters

The binding of a polyphosphazene surface active agent to the substrate can be enhanced by evaporating an adhesion promoter in a reaction chamber using a dynamic vacuum and, if necessary, heat, in the presence of the plasma-activated substrates. The deposition of the adhesion promoter is also carried out inside a plasma chamber, either during or directly after plasma cleaning of the substrate, by introducing the gaseous adhesion promoter into the plasma chamber. To achieve a sufficient vapor pressure of the adhesion promoter, appropriate and correctly dimensioned vacuum pumps are required, for example, a combination of rotary and turbomolecular pumps or other suitable vacuum sources.

Performing a plasma discharge during simultaneous introduction of a reactant gas other than a N₂/O₂ or Ar/O₂ mixture can create a reactive moiety out of an otherwise inert species. Therefore, the reactive nature of adhesion promoting molecules can be enhanced by creating additional anchor sites on the molecule itself. For example, fluoropolymer films can be deposited by plasma excitation of hexafluorobenzene or other fluorine-containing inorganic or organic compounds that would normally be inert in the presence of a substrate. Such polymeric films can improve surface properties for improved adhesion of a polyphosphazene, without the need for adhesion promoters.

### Example 6

### General Procedure for the Deposition of Silane-Based Adhesion Promoters

Silanization protocols can be carried out in the liquid or gas phases. Further, liquid phase procedures can be effected under hydrous or anhydrous conditions, typically employing organic solvents, in which the presence and concentration of water vary. For example, commonly-employed methods for siloxane surface derivatization are carried out either in anhydrous organic solvents or in aqueous organic solvents. In this case, the presence of even trace amounts of water can lead to auto-catalyzed hydrolyzation and subsequent polymerization of the siloxane compounds in the reaction media, in parallel with the surface grafting reaction. Therefore aqueous conditions can lead to siloxane multilayer deposition, while anhydrous reaction media are more preferred in true siloxane monolayer formation.

Reactions in aqueous reaction media are carried out more easily under ambient conditions and typically achieve more complete surface coverage of the siloxane polymer on the substrate. The substrate then is heat-treated which results in a crosslinking of the polymer layer to strengthen adhesion between the polymer and substrate. Based on the previously-employed Stenger silanization process, referenced below, given literature values for film thickness usually vary from a lower limit range from about 4 Å to about 6 Å for a 15 min reaction time, to a range from about 50 Å to about 100 Å for 24-72 h reaction times. Contact angles before crosslinking fall in the range from about 20° to about 30° and rise after crosslinking to the range from about 45° to about 55°. See: Stenger et al., J. Am. Chem. Soc. 1992, 114, 8435-8442; Bascom, W., Macromolecules 1972, 5, 792-798; Heiney et al., Langmuir 2000, 16, 2651-2657; Charles et al., Langmuir 2003, 19, 1586-1591; and White et al., Langmuir 2000, 16, 10471-10481.

Procedures carried out in anhydrous liquid environments come much closer to the theoretically-predicted monolayer thickness of 8.5 Å. If carried out under reflux conditions, a separate crosslinking step can be omitted, and the resulting contact angles are within the range from about 45° to about 55°. Careful and thorough trace water removal can be employed to prevent the polymeric siloxane aggregate formation that can be encountered in aqueous environments. See: Sligar et al., Langmuir 1994, 10, 153-158; Vincent et al (Vandenberg method) Langmuir 1997, 13, 14-22. See also: Langmuir 1996, 12, 4621-4624; Langmuir 1995, 11, 3061-3067; and Haller and Ivan, J. Am. Chem. Soc. 1978, 100, 8050-8055.

Silanization is also carried out in the gas phase. This procedure can achieve the same film quality as an anhydrous liquid phase deposition technique, without risking the formation of polymeric aggregates on the substrate. Whether the procedure is carried out under vacuum or atmospheric conditions, larger polymeric aggregates lack sufficient vapour pressure to be carried over into the gaseous phase; therefore, aggregates are not deposited on the substrate. Moreover, the process of removing physisorbed silanes can be combined with the silanization technique after incubation of the substrates in the silane-enriched environment, prior to crosslinking or exposure to moisture. This process is accomplished by removal of the unreacted silanes under dynamic vacuum. A hybrid between gas phase and liquid phase deposition uses a solvent under reflux to deposit the silanes on the substrate surfaces while achieving similar results without the need for a separate crosslinking step. (See: J. Am. Chem. Soc. 1996, 118, 2950-2953; J. Am. Chem. Soc. 1978, 100, 8050-8055; Haller and Ivan, Langmuir 1993, 9, 2965-2973; Langmuir 1995, 11, 3061-3067).

Thus, as disclosed herein, surface modification of silicone elastomers with adhesion promoters is one preferred procedure for depositing the silanes onto silicone-containing substrates within the context of this invention. However, it is straightforward to deposit a polyphosphazene-specific silane adhesion promoter by any of the aforementioned silanization procedures known in the art.

### Example 7

### Process for Substrate Silanization

Following plasma activation as described above, different silicone substrates were placed in a separate desiccator, and 10 μL-, 50 μL-, or 200 μL-samples of (3-aminopropyl)triethoxysilane (APTES) were placed beneath the substrates in a closed Petri dish. The desiccator was evacuated to a pressure of 1×10⁻¹ mBar, after which the vacuum line was closed to afford a static vacuum. After incubation for 30-60 min in the desiccator, the vacuum valve was opened again to remove the physisorbed silane under a dynamic vacuum, at pressures below about 1×10⁻² mBar. The samples were then heat-treated from about 30 min to about 60 min at 60° C. to crosslink the aminosilane layer. For the polyphosphazene coating evaluation described herein, eight similarly “aminosilanized” silicon wafers were used as reference substrates. After plasma activation, all substrates were silanized in the gas phase, which raised the contact angles for all substrates to the reported literature range of 65-75°.

Other adhesions promoters were also tested and shown to be effective in promoting strong adhesion between a silicone-containing substrate and a polyphosphazene film, specifically a poly[bis(2,2,2-trifluoroethoxy)phosphazene]. Additional adhesion promoters that were tested were: N-methyl-aza-2,2,4-trimethylsilacyclopentane; 2,2-dimethoxy-1,6-diaza-2-silacyclooctane; (3-trimethoxy-silylpropyl)diethylene triamine; and each of the following, for which contact angles are presented:

| | Contact |
|---|---|
| | Angle |
| Adhesion Promoter | CA (H₂O) |
| 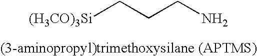 | 120.81 ±4.79° |
| 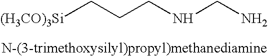 | 119.82 ±4.82° |
| 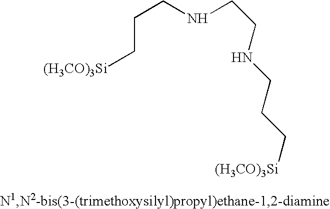 | 114.59 ±0.98° |
| 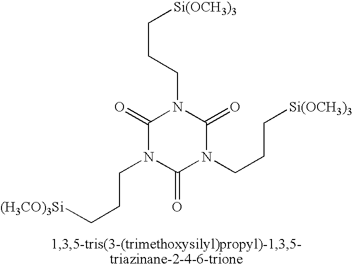 | 115.65 ±0.13° |

### Example 8

### Procedure for Spray Coating a Polyphosphazene Blend

A. Preparation of Substrates. A set of silicone substrates was cut into 2.0 cm×3.6 cm pieces, wiped clean with acetone-moistened, lint-free wipe cloth, rinsed with pure acetone, and blown dry with a stream of argon. These pre-cleaned substrates were transferred into a plasma chamber and plasma-treated at 0.1 mbar for a period of about 8 min. After removing the samples from the chamber, the samples were spray-coated with various (3-aminopropyl)trimethoxysilane (APTMS) adhesion promoter solutions containing poly[bis(2,2,2-trifluoroethoxy)phosphazene] (PzF). These APTMS/PzF spray coating solutions were prepared as provided below.

B. Preparation of Polyphosphazene (PzF) Stock Solution and Dilute Solution. An ethyl acetate (EtOAc)-poly[bis(2,2,2-trifluoroethoxy)phosphazene] (PzF) stock solution was prepared as follows. A 20 g-sample of PzF was combined with 898 g of EtOAc, for a concentration (C) of C=20.0 mg PzF/mL stock solution, 21.8 mg PzF/g stock solution, or 22.2 mg PzF/g EtOAc. This stock solution was diluted as needed with an EtOAc/isoamyl acetate (IAA) mixture to provide a PzF spray coating solution of the desired wt/wt ratio. An EtOAc/IAA mixture with a EtOAc:IAA weight ratio of about 1:1 (wt/wt) typically was used for this purpose. For example, 150 g of the stock (PzF/EtOAc) solution was combined with the EtOAc/IAA mixture that contained 1925 g of EtOAc and 1925 g of IAA to provide a concentration of PzF of C(PzF)=0.82 mg PzF/g spray coating solution.

C. Addition of APTMS/PzF Spray Coating Solutions. Using the dilute PzF solution in EtOAc/IAA, the following (3-aminopropyl)trimethoxysilane (APTMS) spray coating solutions were prepared. The wt % numbers of APTMS are reported as a weight percent APTMS relative to the weight of PzF in that spray coating solution.

1. 1% APTMS/PzF. A spray coating solution was prepared by mixing 4000 g dilute PzF solution and 33.4 mg (32.9 μL) of APTMS. The 4000 g dilute PzF solution was prepared from 150 g stock (PzF/EtOAc) solution, 1925 g EtOAc, 1925 g IAA as provided above. The resulting concentration of APTMS was about 8.2 μL/kg spray coating solution. The resulting concentration of APTMS to PzF was about 1%, that is, relative to the mass of PzF in the prepared spray coating solution.

2. 5% APTMS/PzF. A spray coating solution was prepared by mixing 4000 g dilute PzF solution and 167 mg (164.4 μL) of APTMS as described immediately above, to provide a spray coating solution having a concentration of APTMS of about 41.1 μL/kg spray coating solution. The resulting concentration of APTMS to PzF was about 5%, that is, relative to the mass of PzF in the prepared spray coating solution.

3. 10% APTMS/PzF. A spray coating solution was prepared by mixing 4000 g dilute PzF solution and 334.1 mg (328.8 μL) of APTMS as described above, to provide a spray coating solution having a concentration of APTMS of about 82.2 μL/kg spray coating solution. The resulting concentration of APTMS to PzF was about 10%, that is, relative to the mass of PzF in the prepared spray coating solution.

D. Spray Coating Procedure. For each spray composition, a total amount of 10 mL of the APTMS/PzF spray coating blend was sprayed onto the substrates. The liquid was pumped through a dual feed nozzle using a syringe pump, at a rate of 20 mL/h and nebulized by pressurized Argon at approx. 4 Bar. The distance between each substrate and the spray nozzle was adjusted to 20 cm for each sample. Following application of the APTMS/PzF spray coating, the substrates were placed in a drying oven at 60° C. for about 30 min each to remove residual solvent and to crosslink the APTMS.

E. ASTM Delamination Test. The spray-coated films were placed under an optical microscope and the respective film morphologies evaluated at 2.5×, 5×, and 10× magnification. For performing abrasion experiments, each of the sample films was cut twice at a 90° angle with the scribe tool from an ASTM delamination test kit to get a square 2 mm×2 mm pattern. The test kit used was a Gardco, Model P-A-T Adhesion Test Kit, performing according to ASTM D-3359. The test tape used is a Permacel, P-99, polyester/fiber packaging tape with known specifications. The supplied test tape was placed onto the prepared films, firmly brushed onto the substrate, and after 2 min was peeled off from the film surface. The patterned films were evaluated before and after application of test tape. This test showed that the films sprayed with the 10% APTMS/PzF coating solution showed the largest increase in adhesion. Approximately 90% of the original film surface was intact after removal of the tape. Further, blending the PzF solution with increasing concentrations of APTMS increased the wetting behaviour of the PzF solution and led to continuously smaller granular structures.

F. Film Delamination Tendency. The gradual increase of the APTMS content in the PzF spray coating solution caused an increasing improvement of the adhesion of the PzF films when applying mechanical stress. The first notable difference was observed for PzF solutions containing 5% (wt %) APTMS (in relation to the mass content of PzF in the spraying solution). At 10% concentration, the adhesion was excellent and 90% of the film area remained intact after the application of mechanical stress.

The combination of APTMS and PzF in the spray-coating solution had two beneficial effects. First, it led to an improved wetting ability of the PzF solution on the substrates and decreased detrimental de-wetting effects, thereby smoothing out the PzF film corrugation. As a result, a more homogeneous coating morphology was observed.

Second, the combination of APTMS and PzF in the spray-coating solution greatly increased the adhesion of the deposited PzF films towards the substrate. In comparison to the coating of APTMS monolayer or multilayer substrates with PzF, the adhesion of interfaces obtained by direct blending of an aminosiloxane-forming polymer with PzF resulted in superior adhesion. While not intending to be bound by theory, it is believed that the formation of an interpenetrating network between the two interfaces created a much more extensive surface contact area with more anchoring sites for the film.

The addition of APTMS had no detrimental effects on the overall contact angle of the PzF films, all of which stayed above 90° for all substrates.

### Example 9

### Coating Silicone-Containing Catheters with a Polyphosphazene

Various commercially-available urological catheters were cut into 2 cm segments and coated with a 20 mg/mL PzF solution. One set of samples was used as received, while the other set was pre-treated with an adhesion promoter. Samples were examined by optical microscopy and fluorescence staining. A delamination test was performed after coating. The urinary catheters used (size 14-20 FR, Foley type) are provided in Table 2.

TABLE 2
Silicone-Containing Catheter Samples Used
for Coating with a Polyphoasphazene.

| Manufacturer | Catheter type | Trade Name | Catheter material |
|---|---|---|---|
| Mentor | 16 FR | FOLYSIL | 100% Silicone |
| | Foley | | |
| | 16 FR | FOLATEX | Silicone-coated Latex |
| | Foley | | |
| Kendall/Tyco | 16 FR | ARGYLE | 100% Silicone |
| | Foley | | |
| | 16 FR | KENGUARD | Silicone-coated Latex |
| | Foley | | |
| Rusch/Teleflex | 16 FR | SILKOMED | 100% Silicone |
| | Foley | | |
| | 16 FR | SILKOLATEX | Silicone-coated Latex |
| | Foley | | |
| C R Bard | 16 FR | BARDEX | 100% Silicone |
| | Foley | | |
| | 18 FR | BARDEX | 100% Silicone |
| | Foley | | |
| | 14 FR | BARDIA | Silicone-coated Latex |
| | Foley | | |
| | 16 FR | BARDEX | Silicone-coated Latex |
| | Foley | | |
| | 20 FR | BARDEX | Silicone-coated Latex |
| | Foley | | |
| | 16 FR | SILASTIC | Silicone-coated Latex |
| | Foley | | |

A. Plasma Treatment. Samples were subjected to plasma activation for about 120 sec in a Diener Electronics Femto plasma chamber. The system was evacuated below 5 mbar pressure and normal air was introduced into the chamber as an operating gas, after which the plasma process was initiated. The chamber was vented thereafter and samples were subjected to aminosilanization.

B. Aminosilanization. Samples that had been plasma treated were inserted into a Schlenk tube containing 10 μL APTMS, which was then connected to a standard vacuum line. The vessel was evacuated and held under a dynamic vacuum below 1×10⁻¹ mBar for a period of 60 min. After this time, samples were stored in a drying oven at 65° C. for about 60 min to afford crosslinking of the aminosilane adhesion promoter.

C. Dip Coating. Samples that had been subjected to aminosilanization were then submerged partially into a PzF dip coating solution and withdrawn with a preset speed of 9 mm/min after a short dwelling period of 1 min. The PzF solution was based on OF 282 (11.4×10⁶ gmol⁻¹) dissolved in ethyl acetate.

D. Delamination Tests. Coated samples were immobilized by fixing at the uncoated segment, and the coated tubing section was tightly grasped. The coated section was pulled several (about 4) times through the zone where the pressure was applied.

E. Results. Plasma pretreatment did not cause detectable negative optical changes to the various materials tested, but it did impart a desired increase in surface energy, thereby increasing the tendency of PzF solutions to wet the substrate surface during the coating procedure. Plasma pre-treatment also helped minimize surface contamination prior to handling and provided for surface activation prior to aminosilanization. There was only a marginally-detectable difference between aminosilanized or bare latex substrates, while Silastic® silicone elastomer and silicone materials gained more benefit from the aminosilanization procedure.

Further, it was observed for coating substrates at PzF concentrations below about 5 mg/mL in ethyl acetate, the increase in hydrophobicity of the treated substrates was not substantial. At concentrations above about 5 mg/mL, including at approximately 10 mg/mL and above, typical PzF non-wetting behaviour towards water was observed.

After complete drying of the substrates the sensitive balloon section of all urinary catheters could easily be inflated at moderate pressures (0-1.5 Bar) without causing balloon rupture or PzF film delamination.

Generally, delamination of the PzF films only occurred at the interfacial boundaries of the native and the coated substrate, and only then under a high load of mechanical stress. Under no circumstances was the PzF layer ever observed to became completely detached from either the silicone, Silastic®, or latex substrates.

This example demonstrates that silicone-coated latex catheters can be coated in a straightforward manner without any dewetting effects or lack of PzF adhesion. The coating effect was instantaneously visible in the rise in contact angles against water. Adhesion of the PzF coating under mechanical stress was improved by pre-treatment with APTMS as an adhesion promoter, and the thermal stability of the native substrate required for APTMS crosslinking was sufficient under the conditions employed. It was also observed that catheters made from 100% silicone could be coated in a similar fashion as the latex material, whereas silicone elastomer-based catheter materials such as the C.R.Bard Silastic® Brand took an intermediate position between the latex and pure silicon compounds, in terms of the wetting tendency and PzF adhesion.

This coating study further demonstrated that most commonly available catheter materials could successfully be coated with PzF solutions in ethyl acetate at concentrations above about 10 mg/mL, without causing any discernible damage to the sensitive parts of the catheters. Thus, PzF adhesion was sufficient under the conditions exerted on the catheters, and this adhesion should withstand typical mechanical stress from bending and insertion or removal of the catheters. Catheters made from silicone coated latex, Silastic®, or 100% silicone polymers were therefore well-suited for application of PzF films. The inner lumen of the catheters can be coated in parallel to the outer surface by leaving the drainage holes open during the coating procedure. Additionally, the inflation port of the catheter is not affected by this coating procedure.

### Example 10

### Properties of the PzF-Coated Catheters

Two types of silicone tubing materials (16 French size×11 cm or 20 cm in length), a rubber material made of 100% silicone, and a material containing latex and silicone were evaluated for friction and coating durability. Both types of tubing were coated with PzF according to the method described above. The lubricious property of the PzF coating was evaluated using a FTS5000 Friction Test System (Harland Medical Systems) which allows the measurement of the surface friction and coating durability at the same time by pulling the test sample between two silicone rubber pads clamped at a programmable force. Fifteen cycle times were applied to each test sample, and a clamping force of 300 g was used in this testing. An average pull forced of the 15 cycle runs were recorded.

The results showed there was no PzF-delamination observed on either the silicone or the silicone/latex coated-tubing samples. Preliminary results of the average pulled forces are summarized in Table 3 below:

TABLE 3
Lubricious Property of PzF-Coated as
Compared to Uncoated Control Tubing

| | Average | Average |
|---|---|---|
| | Pulled Force | Friction |
| Tubing Samples | (g ± SD) | Force |
| Silicone-Coated | 348.2 ± 44.7 | 1.161 |
| Silicone- | 460.4 ± 32.0 | 1.535 |
| Uncoated | | |
| Latex/Silicone- | 342.7 ± 10.0 | 1.142 |
| Coated | | |
| Latex/Silicone - | 475.0 ± 0 | 1.583 |
| Uncoated | | |
| Latex/Silicone - | 567.9 ± 10.04 | 1.893 |
| Coated | | |
| Latex/Silicone | 689.3 ± 25.24 | 2.298 |
| Uncoated | | |

These results demonstrate both the silicone catheters and the silicone/latex catheters that were coated with PzF had significantly reduced friction forces and therefore a significantly enhanced lubricity.

### Example 10

### Biological Evaluation of PzF-Coated Silicone Tubing

### Bacterial Adhesion and Biofilm Formation

Two types of silicone tubing materials were evaluated, a Silastic® material and a material containing latex and silicone. Approximately 30 cm and 16 French sizes of both types of tubing were coated with PzF according to the method described above. Both samples were evaluated for bacterial adhesion and biofilm formation using an artificial urine medium containing E. coli. This evaluation utilized two separated testing methods: a) a dynamic, continuous flow method; and b) a static method or segmented test, as described below.

A. Continuous Flow Testing. Each sample of PzF-coated or uncoated tubing was installed into each channel of a test system consisting of four parallel channels (one channel per tubing). The entire system was placed into a 37° C. incubator and allowed to equilibrate with a continuous flow of an artificial urine at least 30 minutes, before inoculation of Escherichia coli (ATCC 25922), previously grown in artificial urine medium at 37° C. The flow of artificial urine medium was maintained at a rate of approximately 0.7 mL/min for up to 7 days. A segment of approximately 5.0 cm was cut from the downstream end of the tubing sample, at a designated time interval of 1, 3, and 7 days. The 5 cm pieces were divided into 3 portions which were analyzed for bacterial adhesion by plate count, biofilm formation by SEM, and viable cells assessment by Confocal Laser Scanning Microscopy (CSLM) after staining bacteria with LIVE/DEAD® Baclight™ bacterial viability kit (L7012, Molecular Probes, Oregon, USA). The following continuous flow test results were obtained.

Flow Test Plate Count Analysis. The results of the plate count analysis are summarized in Table 4 below. A rinsing step using PBS was applied to the day-7 samples to remove unattached cells before analyzing for plate counts. These results indicate biofilms that formed on the coated catheters were not adhered to the catheters in contrast to the biofilms that formed on the uncoated catheters.

TABLE 4
Viable Cell Counts per cm² × 10⁶

| Tubing Samples | Day 1 | Day 3 | Day 7 - Rinsed |
|---|---|---|---|
| Silastic ®-uncoated | 13.5 | 45.5 | 9.68 |
| Silastic ®-coated | 8.33 | 25.4 | 0.03 |
| Silicone-uncoated | 10.1 | 16.7 | 4.03 |
| Silicone-coated | 4.88 | 1.81 | 0.16 |

Flow Test Confocal and SEM Analysis. Representative confocal and SEM images of coated and uncoated samples showed less biofilm present on the coated catheters relative to the uncoated controls discussed above. Consistent with the data in Table 4, there were significantly more live cells present on the surfaces of the uncoated catheters than on the coated catheters.

B. Segmented, Static Mode Test. This test utilized 3 cm tubing segments. Only Silastic® segmented samples, coated and uncoated, were used. Sample were placed in test tubes containing artificial urine inoculated with E. coli as described above. A set of triplicate segmented samples (3×3 cm) was removed at 4 different times, specifically at 2 hr, 24 hr, 48 hr, and 72 hr following exposure to the urine medium at 37° C. containing E. coli. Samples were tested for bacterial adhesion by plate counts and viable cells assessment by Confocal Laser Scanning Microscopy (CSLM). For static test plate counts, three uncoated and three coated segments from each time point were scraped and spread plated in triplicate for viable (culturable) cell counts. For static test CSLM analysis, sections of uncoated and coated samples were stained with LIVE/DEAD® Baclight™ bacterial viability kit according to manufacturer's instructions (L7012, Molecular Probes, Oregon, USA). The following static mode test results were obtained.

Static Test Plate Counts and Confocal Analysis. The results summarized in Table 5 indicated reduction in E. coli binding to PzF-coated samples compared to the corresponding uncoated sample. A dramatic reduction in the cell counts was observed after 2 hours of bacterial exposure. These results show a finding consistent with the flow testing method. Also consistent with the flow test, there were significantly more live cells present on the surfaces of the uncoated Silastic® samples than on the coated Silastic samples.

TABLE 5
Viable Cell Counts/m² of PzF-Coated Silastic ® Samples

| Tubing | | | | |
|---|---|---|---|---|
| Samples | 2 hours | Day 1 | Day 3 | Day 7 |
| Silastic ®- | 3600 | 2.0 × 10⁶ | 5.8 × 10⁶ | 1.8 × 10⁶ |
| uncoated | | | | |
| Silastic ®- | 28 | 4.5 × 10⁵ | 9.9 × 10⁵ | 1.1 × 10⁶ |
| coated | | | | |

1. A medical device comprising a polyorganosiloxane in combination with a polyphosphazene, wherein
the polyphosphazene has the formula: 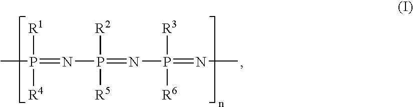
n is 2 to ∞; and
R¹ to R⁶ are each selected independently from alkyl, aminoalkyl, haloalkyl, thioalkyl, thioaryl, alkoxy, haloalkoxy, aryloxy, haloaryloxy, alkylthiolate, arylthiolate, alkylsulphonyl, alkylamino, dialkylamino, heterocycloalkyl comprising one or more heteroatoms selected from nitrogen, oxygen, sulfur, phosphorus, or a combination thereof, or heteroaryl comprising one or more heteroatoms selected from nitrogen, oxygen, sulfur, phosphorus, or a combination thereof.

2. The medical device according to claim 1, wherein at least one of R¹ to R⁶ is an alkoxy group substituted with at least one fluorine atom.

3. The medical device according to claim 1, wherein at least one of R¹ to R⁶ is selected from OCH₃, OCF₃, OCH₂CH₃, OCH₂CF₃, OCH₂CH₂CH₃, OCH₂CH₂CF₃, OCH₂CF₂CF₃, OCH(CF₃)₂, OCCH₃(CF₃)₂, OCH₂CF₂CF₂CF₃, OCH₂(CF₂)₃CF₃, OCH₂(CF₂)₄CF₃, OCH₂(CF₂)₅CF₃, OCH₂(CF₂)₆CF₃, OCH₂(CF₂)₇CF₃, OCH₂CF₂CHF₂, OCH₂CF₂CF₂CHF₂, OCH₂(CF₂)₃CHF₂, OCH₂(CF₂)₄CHF₂, OCH₂(CF₂)₅CHF₂, OCH₂(CF₂)₆CHF₂, or OCH₂(CF₂)₇CHF₂.

4. A medical device according to claim 1, wherein the polyphosphazene is poly[bis(2,2,2-trifluoroethoxy)]phosphazene.

5. A medical device according to claim 1, wherein the polyorganosiloxane is coated with, reacted with, blended with, grafted to, bonded to, crosslinked with, copolymerized with, or coated and/or reacted with an intermediate layer that is coated and/or reacted with the polyphosphazene.

6. A medical device according to claim 1, wherein the polyorganosiloxane is coated with the polyphosphazene, wherein the polyphosphazene coating has a thickness from about one polymer monolayer to about 100 μm.

7. A medical device according to claim 1, further comprising a tie layer between the polyorganosiloxane and the polyphosphazene.

8. A medical device according to claim 1, wherein the polyorganosiloxane is contacted with an adhesion promoter selected from N-methyl-aza-2,2,4-trimethylsilacyclopentane; 2,2-dimethoxy-1,6-diaza-2-silacyclooctane; (3-trimethoxysilylpropyl)diethylene triamine; (3-aminopropyl)trimethoxysilane (APTMS); N-(3-(trimethoxysilyl)propyl)methanediamine; N¹,N²-bis(3-(trimethoxysilyl)propyl)ethane-1,2-diamine; 1,3,5-tris(3-(trimethoxysilyl)propyl)-1,3,5-triazinane-2-4-6-trione; or any combination thereof, prior to combining the polyorganosiloxane with the polyphosphazene.

9. A medical device according to claim 1, wherein the polyorganosiloxane is functionalized with a functional moiety selected from hydroxy, carboxy, carboxyl, aldehyde, peroxy, amino, imino, halo, hydride, nitro, alkoxy, alkylsulfonyl, dialkyl amino, aryloxy, N-heterocycloalkyl, N-heteroaryl, monoethylene imine, oligoethylene imine, polyethylene imine, fluoride, chloride, bromide, iodide, cyclic polyphosphazene, monosilane, oligosilane, polysilane, amino-terminated silane, amino-terminated alkene, nitro-terminated alkene, alkylphosphonic acid, ureido-terminated silane, glycidyl-terminated silane, thiol-terminated silane, acroyl-terminated silane, perfluorosilane, or a combination thereof prior to combining with the polyphosphazene.

10. A medical device according to claim 1, wherein the polyorganosiloxane is contacted with an adhesion promoter, a swelling agent, a crosslinking agent, an acid, a base, an oxidizing agent, a fluorination agent, a reducing agent, an X-ray source, actinic radiation, ionizing radiation, e-beam radiation, corona discharge, flame pyrolysis, plasma discharge, or any combination thereof, prior to combining with the polyphosphazene.

11. A medical device according to claim 1, wherein the polyphosphazene has a molecular weight of at least about 70,000 g/mol.

12. A medical device according to claim 1, wherein the polyorganosiloxane is selected from the classification MQ, VMQ, PMQ, PVMQ, or FVMQ, in accordance with ASTM D1418.

13. A method for making a medical device, comprising:
a. providing a medical device that comprises a polyorganosiloxane; and
b. combining the polyorganosiloxane with a polyphosphazene;
wherein
the polyphosphazene has the formula: 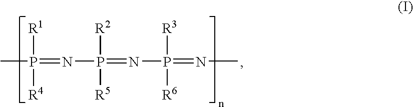
n is 2 to ∞; and
R¹ to R⁶ are each selected independently from alkyl, aminoalkyl, haloalkyl, thioalkyl, thioaryl, alkoxy, haloalkoxy, aryloxy, haloaryloxy, alkylthiolate, arylthiolate, alkylsulphonyl, alkylamino, dialkylamino, heterocycloalkyl comprising one or more heteroatoms selected from nitrogen, oxygen, sulfur, phosphorus, or a combination thereof or heteroaryl comprising one or more heteroatoms selected from nitrogen, oxygen, sulfur, phosphorus, or a combination thereof.

14. The method for making a medical device according to claim 13, wherein at least one of R¹ to R⁶ is an alkoxy group substituted with at least one fluorine atom.

15. The method for making a medical device according to claim 13, wherein at least one of R¹ to R⁶ is selected from OCH₃, OCF₃, OCH₂CH₃, OCH₂CF₃, OCH₂CH₂CH₃, OCH₂CH₂CF₃, OCH₂CF₂CF₃, OCH(CF₃)₂, OCCH₃(CF₃)₂, OCH₂CF₂CF₂CF₃, OCH₂(CF₂)₃CF₃, OCH₂(CF₂)₄CF₃, OCH₂(CF₂)₅CF₃, OCH₂(CF₂)₆CF₃, OCH₂(CF₂)₇CF₃, OCH₂CF₂CHF₂, OCH₂CF₂CF₂CHF₂, OCH₂(CF₂)₃CHF₂, OCH₂(CF₂)₄CHF₂, OCH₂(CF₂)₅CHF₂, OCH₂(CF₂)₆CHF₂, or OCH₂(CF₂)₇CHF₂.

16. The method for making a medical device according to claim 13, wherein the polyphosphazene is poly[bis(2,2,2-trifluoroethoxy)]phosphazene.

17. The method for making a medical device according to claim 13, wherein the polyorganosiloxane is coated with, reacted with, blended with, grafted to, bonded to, crosslinked with, copolymerized with, or coated and/or reacted with an intermediate layer that is coated and/or reacted with the polyphosphazene.

18. The method for making a medical device according to claim 13, wherein the polyorganosiloxane is coated with the polyphosphazene, wherein the polyphosphazene coating has a thickness from about one polymer monolayer to about 100 μm.

19. The method for making a medical device according to claim 13, further comprising contacting the polyorganosiloxane with an adhesion promoter selected from N-methyl-aza-2,2,4-trimethylsilacyclopentane; 2,2-dimethoxy-1,6-diaza-2-silacyclooctane; (3-trimethoxysilylpropyl)diethylene triamine; (3-aminopropyl)trimethoxysilane (APTMS); N-(3-(trimethoxysilyl)propyl)methanediamine; N¹,N²-bis(3-(trimethoxysilyl)propyl)ethane-1,2-diamine; 1,3,5-tris(3-(trimethoxysilyl)propyl)-1,3,5-triazinane-2-4-6-trione; or any combination thereof; prior to combining the polyorganosiloxane with the polyphosphazene.

20. The method for making a medical device according to claim 13, further comprising functionalizing the polyorganosiloxane with a functional moiety selected from hydroxy, carboxy, carboxyl, aldehyde, peroxy, amino, imino, halo, hydride, nitro, alkoxy, alkylsulfonyl, dialkyl amino, aryloxy, N-heterocycloalkyl, N-heteroaryl, monoethylene imine, oligoethylene imine, polyethylene imine, fluoride, chloride, bromide, iodide, cyclic polyphosphazene, monosilane, oligosilane, polysilane, amino-terminated silane, amino-terminated alkene, nitro-terminated alkene, alkylphosphonic acid, ureido-terminated silane, glycidyl-terminated silane, thiol-terminated silane, acroyl-terminated silane, perfluorosilane, or a combination thereof, prior to combining the polyorganosiloxane with the polyphosphazene.

21. The method for making a medical device according to claim 13, further comprising contacting the polyorganosiloxane with an adhesion promoter, a swelling agent, a crosslinking agent, an acid, a base, an oxidizing agent, a fluorination agent, a reducing agent, an X-ray source, actinic radiation, ionizing radiation, e-beam radiation, corona discharge, flame pyrolysis, plasma discharge, or any combination thereof, prior to combining the polyorganosiloxane with the polyphosphazene.

22. The method for making a medical device according to claim 13, wherein the polyphosphazene has a molecular weight of at least about 70,000 g/mol.

23. A method for making a medical device, comprising:
a. providing a medical device that comprises a polyorganosiloxane;
b. optionally, cleaning the surface of the polyorganosiloxane;
c. contacting the polyorganosiloxane with an adhesion promoter selected from N-methyl-aza-2,2,4-trimethylsilacyclopentane; 2,2-dimethoxy-1,6-diaza-2-silacyclooctane; (3-trimethoxysilylpropyl)diethylene triamine; (3-aminopropyl)trimethoxysilane (APTMS); N-(3-(trimethoxysilyl)propyl)methanediamine; N¹,N²-bis(3-(trimethoxysilyl)propyl)ethane-1,2-diamine; 1,3,5-tris(3-(trimethoxysilyl)propyl)-1,3,5-triazinane-2-4-6-trione; or any combination thereof; and
d. contacting the polyorganosiloxane with poly[bis(2,2,2-trifluoroethoxy)]phosphazene at substantially the same time or after the polyorganosiloxane is contacted with the adhesion promoter.

24. The method for making a medical device according to claim 23, wherein cleaning the surface of the polyorganosiloxane occurs by plasma activation or contacting the polyorganosiloxane with a basic solution optionally comprising a swelling agent.

25. A method for improving the biocompatibility of a medical device when in contact with tissue or fluids of a mammal, comprising:
a. providing a medical device that comprises a polyorganosiloxane;
b. optionally, cleaning the surface of the polyorganosiloxane;
c. contacting the polyorganosiloxane with an adhesion promoter selected from N-methyl-aza-2,2,4-trimethylsilacyclopentane; 2,2-dimethoxy-1,6-diaza-2-silacyclooctane; (3-trimethoxysilylpropyl)diethylene triamine; (3-aminopropyl)trimethoxysilane (APTMS); N-(3-(trimethoxysilyl)propyl)methanediamine; N¹,N²-bis(3-(trimethoxysilyl)propyl)ethane-1,2-diamine; 1,3,5-tris(3-(trimethoxysilyl)propyl)-1,3,5-triazinane-2-4-6-trione; or any combination thereof; and
d. contacting the polyorganosiloxane with poly[bis(2,2,2-trifluoroethoxy)]phosphazene at substantially the same time or after the polyorganosiloxane is contacted with the adhesion promoter;
wherein the surface of the medical device that is in contact with tissue or fluids of the mammal comprises the polyorganosiloxane.
